(19) United States
(12) Patent Application Publication
(10) Pub. No.: US 2026/0325603 A1
(43) Pub. Date: Oct. 22, 2026

(54) STABLE CYCLOSPORINE OPHTHALMIC FORMULATION AND MANUFACTURING PROCESS THEREOF

(71) Applicant: Sun Pharmaceutical Industries Limited, Mumbai, Maharashtra (IN)

(72) Inventors: Ajay Jaysingh KHOPADE, Vadodara, Gujarat (IN); Arindam HALDER, Vadodara, Gujarat (IN)

(21) Appl. No.: 19/486,600

(22) PCT Filed: May 29, 2024
(86) PCT No.: PCT/IB2024/055222

§ 371 Date: Nov. 21, 2025

(30) Foreign Application Priority Data
May 29, 2023 (IN) 202321036901

(51) Int. Cl. A61K 38/13; A61K 9/107; A61K 47/02; A61K 47/10; A61K 47/32

(52) U.S. Cl. CPC A61K 38/13; A61K 9/1075; A61K 47/02; A61K 47/10; A61K 47/32

(57) ABSTRACT

The present invention relates to a stable nanomicellar ophthalmic formulation comprising cyclosporine, hydrogenated 40 polyoxyl castor oil, octoxynol-40, and an aqueous vehicle, wherein the stable nanomicellar ophthalmic formulation is made by a method comprising: a) mixing the hydrogenated 40 polyoxyl castor oil and the octoxynol-40, wherein the mixing produces a Mixture A comprising a water content of less than 3%; b) adding the cyclosporine to the Mixture A to form a Mixture B; and c) mixing the Mixture B with the aqueous vehicle, thereby forming the stable nanomicellar ophthalmic formulation.

### FIELD OF THE INVENTION

The present invention relates to a stable nanomicellar ophthalmic formulation comprising cyclosporine, hydrogenated 40 polyoxyl castor oil, octoxynol-40, and an aqueous vehicle, wherein the stable nanomicellar ophthalmic formulation is made by a method comprising: a) mixing the hydrogenated 40 polyoxyl castor oil and the octoxynol-40, wherein the mixing produces a Mixture A comprising a water content of less than 3%; b) adding the cyclosporine to the Mixture A to form a Mixture B; and c) mixing the Mixture B with the aqueous vehicle, thereby forming the stable nanomicellar ophthalmic formulation.

### BACKGROUND OF THE INVENTION

Cyclosporine nanomicellar ophthalmic solutions are generally described in U.S. Pat. No. 10,918,694, wherein the ophthalmic solution comprises 0.087-0.093 wt % cyclosporine, a polyoxyl lipid or fatty acid and a polyalkoxylated alcohol. In some embodiments, the solution comprises 0.087-0.093 wt % cyclosporine, 0.5-5 wt % of one or more polyoxyl lipids or fatty acids selected from HCO-40, HCO-60, HCO-80 and HCO-100; and about 0.01-0.1 wt % octoxynol-40. U.S. Pat. No. 10,918,694 further describes methods of preparing such cyclosporine solutions.

### SUMMARY OF THE INVENTION

In some embodiments, the present disclosure provides a stable nanomicellar ophthalmic formulation comprising cyclosporine, hydrogenated 40 polyoxyl castor oil, octoxynol-40, and an aqueous vehicle, wherein the stable nanomicellar ophthalmic formulation is made by a method comprising:
a) mixing the hydrogenated 40 polyoxyl castor oil and the octoxynol-40 at a temperature of 100° C. or above, wherein the mixing produces a Mixture A comprising a water content of less than 3%;
b) adding the cyclosporine to the Mixture A to form a Mixture B; and
c) mixing the Mixture B with the aqueous vehicle, thereby forming the stable nanomicellar ophthalmic formulation.

In some embodiments, the disclosure provides a stable nanomicellar ophthalmic formulation comprising: i) a concentrate comprising cyclosporine, hydrogenated 40 polyoxyl castor oil, and octoxynol-40; and ii) an aqueous vehicle, wherein the concentrate is made by a method comprising:
a) mixing the hydrogenated 40 polyoxyl castor oil and the octoxynol-40 at a temperature of 100° C. or above, wherein the mixing produces a Mixture A comprising a water content of less than 3%; and
b) adding the cyclosporine to the Mixture A to form a Mixture B, wherein the Mixture B comprises a water content of less than 3%. In some embodiments, the formulation is made by mixing the concentrate with the aqueous vehicle.

In some embodiments, the disclosure provides a method of making a stable nanomicellar ophthalmic formulation, comprising:
a) mixing hydrogenated 40 polyoxyl castor oil and octoxynol-40 at a temperature of 100° C. or above, wherein the mixing produces a Mixture A comprising a water content of less than 3%;
b) adding cyclosporine to the Mixture A to form a Mixture B; and
c) mixing the Mixture B with the aqueous vehicle, thereby forming the stable nanomicellar ophthalmic formulation. In some embodiments, the octoxynol-40 comprises less than 20 ppm total impurities.

In some embodiments, the disclosure provides a method of making a stable nanomicellar ophthalmic formulation, comprising:
a) mixing hydrogenated 40 polyoxyl castor oil and octoxynol-40, wherein the octoxynol-40 comprises less than 3% impurities and a water content of less than 10%, wherein the mixing produces a Mixture A comprising a water content of less than 3%;
b) adding cyclosporine to the Mixture A to form a Mixture B; and
c) mixing the Mixture B with the aqueous vehicle, thereby forming the stable nanomicellar ophthalmic formulation.

In some embodiments, the mixing of a) is at a temperature above 55° C. In some embodiments, the mixing of a) is at a temperature above 100° C. In some embodiments, the octoxynol-40 comprises less than 10 ppm total impurities. In some embodiments, the octoxynol-40 comprises less than 5 ppm total impurities.

In some embodiments, the method makes at least 20 L of the formulation. In some embodiments, the method makes at least 100 L of the formulation. In some embodiments, the method makes at least 1000 L of the formulation.

In some embodiments, the Mixture B comprises a water content below about 3%. In some embodiments, the water content of Mixture A and/or the water content of Mixture B is below about 2%. In some embodiments, the water content of Mixture A and/or the water content of Mixture B is below about 1%. In some embodiments, the water content of Mixture A and/or the water content of Mixture B is below about 0.5%.

In some embodiments, the mixing of a) is at a temperature of about 100° C. to about 200° C. In some embodiments, the mixing of a) is at a temperature of about 110° C. to about 160° C. In some embodiments, the mixing of a) is at a temperature of about 120° C. to about 140° C. In some embodiments, the mixing of a) is at a temperature of about 127° C. to about 130° C. In some embodiments, the mixing of a) is at a temperature of about 130° C. or above.

In some embodiments, the mixing of a) is performed for about 10 minutes to about 90 minutes. In some embodiments, the mixing of a) is performed for about 20 minutes to about 60 minutes. In some embodiments, the mixing of a) is performed for about 30 minutes to about 40 minutes. In some embodiments, the mixing of a) is performed at about 100 to about 800 rpm. In some embodiments, the mixing of a) is performed at about 130 to about 180 rpm.

In some embodiments, step b) is performed at a temperature of 55° C. or above. In some embodiments, step b) is performed at a temperature of 100° C. or above. In some embodiments, step b) is performed at a temperature of 130° C. or above. In some embodiments, step b) is performed at the same temperature as the mixing of a). In some embodiments, step b) further comprises mixing until the cyclosporine is completely dissolved.

In some embodiments, the aqueous vehicle comprises Water for Injection (WFI). In some embodiments, the method further comprises, following step c), adding a buffer, a tonicity agent, a bioadhesive agent, a pH adjusting agent, or combination thereof. In some embodiments, the buffer comprises phosphate buffer; the tonicity agent comprises sodium chloride, the bioadhesive agent comprises povidone; and the pH adjusting agent comprises sodium hydroxide and/or hydrochloric acid.

In some embodiments, the method further comprises, following step c): adding in the following order: i) sodium phosphate monobasic; ii) sodium phosphate dibasic; iii) sodium chloride; and iv) povidone. In some embodiments, the method further comprises, following iv): v) adjusting pH to about 6.5 to about 7.2 with sodium hydroxide and/or hydrochloric acid.

In some embodiments, the stable nanomicellar ophthalmic formulation comprises: 0.09 wt % cyclosporine; about 1.0 wt % hydrogenated 40 polyoxyl castor oil; and about 0.05 wt % octoxynol-40. In some embodiments, the stable nanomicellar ophthalmic formulation comprises: 0.09 wt % cyclosporine; about 1.0 wt % hydrogenated 40 polyoxyl castor oil; about 0.05 wt % octoxynol-40; about 0.05 wt % sodium chloride; about 0.3 wt % povidone; phosphate buffer; and sodium hydroxide and/or hydrochloric acid.

In some embodiments, the phosphate buffer comprises sodium phosphate monobasic and/or sodium phosphate dibasic. In some embodiments, the formulation comprises about 0.20 wt % to about 0.55 wt % sodium phosphate monobasic and about 0.23 wt % to about 0.47 wt % sodium phosphate dibasic. In some embodiments, osmolality of the formulation is about 100 to about 300 mOsmol/kg. In some embodiments, osmolality of the formulation is about 150 to about 200 mOsmol/kg. In some embodiments, pH of the formulation is about 6.5 to about 7.2.

In some embodiments, the density of the formulation is about 1.0 to about 1.5 g/ml. In some embodiments, the surface tension of the formulation is about 30 mN/m to about 40 mN/m. In some embodiments, the drop size of the formulation is about 20 μL to about 40 μL. In some embodiments, the particle size Z_{avg} of the formulation is about 10 nm to about 20 nm. In some embodiments, the zeta potential of the formulation is about 0 mV. In some embodiments, the formulation has about 100% cyclosporine encapsulation efficiency. In some embodiments, the cloud point of the formulation is about 40° C. to about 45° C.

In some embodiments, the cyclosporine comprises any one of forms (i)-(iv): (i) an amorphous form; (ii) a form with characteristic XRD peaks at 2-theta (deg.) 6.9, 7.8, 9.4 and 15.9; (iii) a form with characteristic XRD peaks at 2-theta (deg.) 7.4, 8.7, 14.4 and 17.5; and (iv) a form with characteristic XRD peaks at 2-theta (deg.) 8.5, 9.3, 11.6 and 20.3.

In some embodiments, the formulation comprises less than about 2 wt % of an impurity characterized by a relative retention time (RRT) of about 2.0 to about 2.7.

### BRIEF DESCRIPTION OF THE DRAWINGS

The following drawings form part of the specification and are included to further demonstrate exemplary embodiments of certain aspects of the invention.

FIG. 1 shows a representative Cryogenic Transmission Electron Microscopy (Cryo TEM) image of a composition described in embodiments herein.

FIG. 2 shows a representative Cryo TEM image of a composition described in embodiments herein.

FIG. 3 shows a representative Cryo TEM image of a composition described in embodiments herein.

FIG. 4 shows a representative Differential Scanning calorimetry (DSC) scan for a composition described in embodiments herein.

FIG. 5 shows a representative Differential Scanning calorimetry (DSC) scan for a composition described in embodiments herein.

### DETAILED DESCRIPTION OF THE INVENTION

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” means that the specific terms recited are representative examples and embodiments of the disclosure that are not intended to be limited to the specific examples referenced or cited unless explicitly stated otherwise.

As used herein, “about” can mean plus or minus 10% of the provided value. Where ranges are provided, they are inclusive of the boundary values. “About” can additionally or alternately mean either within 10% of the stated value, or within 5% of the stated value, or in some cases within 2.5% of the stated value; or, “about” can mean rounded to the nearest significant digit.

As used herein, “between” is a range inclusive of the ends of the range. For example, a number between x and y explicitly includes the numbers x and y and any numbers that fa 1 within x and y.

Unless otherwise indicated, a l percentages of components of a component described herein are by weight of the composition, also expressed as “% wt” or “wt %.”

As used herein, the “water content” of a composition refers to the percentage by weight of water present in the composition.

As used herein, the term “micelle” or “nanomicelle” refers to an aggregate or cluster of surfactant molecules. micelles only form when the concentration of surfactant is greater than the critical micelle concentration (CMC). Surfactants are chemicals that are amphipathic, which means that they contain both hydrophobic and hydrophilic groups. Micelles can exist in different shapes, including spherical, cylindrical, and discoidal. A micelle comprising at least two different molecular species is a mixed micelle. In some embodiments, formulations of the present disclosure include an aqueous, clear, mixed micellar or mixed nanomicellar solution. In some embodiments, the formulation includes, but is not limited to, a mixed nanomicellar solution as described in U.S. Pat. No. 10,918,694. In some embodiments, the formulation comprises a drug, e.g., cyclosporine, incorporated and/or encapsulated in micelles that are dispersed in an aqueous medium.

Cyclosporine, also known as “cyclosporine A,” “ciclosporin” or “cyclosporin,” was originally extracted from the soil fungus Potypaciadium inflatum (also known as Tolypocladium inflatum) and has a cyclic 11-amino acid structure. Cyclosporine binds to the cytosolic protein cyclophilin of immunocompetent lymphocytes, especially T-lymphocytes, forming a complex. The complex inhibits calcineurin, which under normal circumstances induces the transcription of interleukin-2 (IL-2). Cyclosporine also inhibits lymphokine production and interleukin release, leading to a reduced function of effector T-cells. Cyclosporine is approved in the U.S. for treating of rheumatoid arthritis and psoriasis, persistent nummular keratitis following adenoviral keratoconjunctivitis, and as eye drops for treating dry eyes caused by Sjögren's syndrome and meibomian gland dysfunction.

Different forms of cyclosporine show different solubility and stability. Cyclosporine with characteristic x-ray diffraction (XRD) peaks at 2-theta (deg.) 6.9, 7.8, 9.4 and 15.9 (referred to herein as “Cs Form 1”) is the most soluble form of cyclosporine and is useful in the preparation of solution formulations. However, this is not the most stable form and may convert to the less soluble forms of cyclosporine, thus affecting the stability of the solution. Cs Form 1 may convert to a more stable and less soluble cyclosporine with characteristic XRD peaks at 2-theta (deg.) 7.4, 8.7, 14.4 and 17.5 (referred to herein as “Cs Form 2”) during dissolution of cyclosporine in surfactants at 55-60° C. Cs Form 1 may also convert to another less soluble form of cyclosporine with characteristic XRD peaks at 2-theta (deg.) 8.5, 9.3, 11.6 and 20.3 (referred to herein as “Cs Form 3”). Further, the amorphous form of cyclosporine, which is also one of the more soluble forms and useful in the preparation of solution formulations, may recrystallize to these two less soluble forms, Cs Form 2 and Cs Form 3.

The conversion of cyclosporine from the more soluble forms, e.g., Cs Form 1 or amorphous form, to the less soluble forms Cs Form 2 and Cs Form 3, is facilitated by several factors including but not limited to moisture and solvent temperature. This conversion also depends on stresses such as high temperature, long term storage, and the like. Thus, the manufacturing process for a cyclosporine formulation requires modification depending on the form of cyclosporine used. For example, for the amorphous form and Cs Form 1, wetting followed by complete dissolution at the temperature up to 70° C. is required. However, if Cs Form 2 and Cs Form 3 are used, temperatures as high as 130° C. are needed for complete dissolution.

Further, the formulation must be manufactured with tight control of API specification, process temperature, and time to avoid instability and interconversion of cyclosporine forms. It is also rather difficult to identify to what extent this conversion has taken place before completing the manufacturing process of the cyclosporine formulation. This may lead to formation of seeds within the composition either at the initial stage of the manufacturing process or during storage at higher temperatures, which later crystallizes from the drug product and renders the product unusable. The soft mesophasic or liquid crystalline form of cyclosporine formed as intermediate in the non-aqueous phase may be responsible for such instability.

In some embodiments, the disclosure provides a stable cyclosporine formulation and method of its preparation that prevents conversion to less soluble forms of cyclosporine during the manufacturing process and during storage. In some embodiments, the disclosure provides a stable nanomicellar ophthalmic formulation and an improved method of making such stable formulation. In some embodiments, the method for making the formulation results in a stable formulation irrespective of any form of cyclosporine being used in the formulation. In some embodiments, the method does not lead to conversion of one cyclosporine form to another. In some embodiments, the method does not lead to conversion of a soluble form of cyclosporine to the less soluble forms of cyclosporine and further prevents precipitation of cyclosporine in the formulation during long-term stability.

In some embodiments, the disclosure provides a stable nanomicellar ophthalmic formulation comprising cyclosporine, a polyoxyl lipid or fatty acid, a polyalkoxylated alcohol, and an aqueous vehicle.

In some embodiments, the disclosure provides a stable nanomicellar ophthalmic formulation comprising: i) a concentrate comprising cyclosporine, a polyoxyl lipid or fatty acid, and a polyalkoxylated alcohol; and ii) an aqueous vehicle.

As used herein, the term “polyoxyl lipid or fatty acid” refers to mono- and diesters of lipids or fatty acids and polyoxyethylene diols. Polyoxyl lipids or fatty acids may be numbered (“n”) according to the average polymer length of the oxyethylene units (e.g., 40, 60, 80, 100). The term “n>40 polyoxyl lipid” means that the polyoxyl lipid or fatty acid has an average oxyethylene polymer length equal to or greater than 40 units. Stearate hydrogenated castor oil and castor oil are common lipids/fatty acids commercially available as polyoxyl lipids or fatty acid; however, it is understood that any lipid or fatty acid can be polyoxylated to become a polyoxyl lipid or fatty acid as contemplated herein. Examples of polyoxyl lipid or fatty acids include, without limitation, hydrogenated polyoxyl castor oils such as HCO-40, HCO-60, HCO-80, HCO-100, polyoxyl 40 stearate, and polyoxyl 35 castor oil. In some embodiments, the polyoxyl lipid or fatty acid is a polyoxyl lipid. In some embodiments, the polyoxyl lipid or fatty acid is hydrogenated polyoxyl castor oil. In some embodiments, the polyoxyl lipid or fatty acid is hydrogenated 40 polyoxyl castor oil (HCO-40).

In some embodiments, the polyoxyl lipid or fatty acid, e.g., HCO-40, is about 0.05 to about 5%, or about 0.1% to about 4.5%, or about 0.2% to about 4%, or about 0.3% to about 3.5%, or about 0.4% to about 3%, or about 0.5% to about 2.5%, or about 0.6% to about 2%, or about 0.7% to about 1.5%, or about 0.8% to about 1.2%, or about 1% by weight of the formulation. In some embodiments, the polyoxyl lipid or fatty acid, e.g., HCO-40, is about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, or about 2.0% by weight of the formulation.

As used herein, the term “polyalkoxylated alcohol” refers to a fatty alcohol ethoxylated and/or a fatty acid propoxylated with various degrees of alkoxylation. In some embodiments, the polyalkoxylated alcohol is octoxynol. In some embodiments, the polyalkoxylated alcohol is octoxynol-40.

In some embodiments, the polyalkoxylated alcohol, e.g., octoxynol-40, is about 0.001% to about 5%, or about 0.005% to about 4%, or about 0.01% to about 3%, or about 0.02% to about 2%, or about 0.03% to about 1%, or about 0.04% to about 0.07%, or about 0.05% by weight of the formulation. In some embodiments, the polyalkoxylated alcohol, e.g., octoxynol-40, is about 0.001%, about 0.005%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, or about 0.5% by weight of the formulation.

In some embodiments, the formulation comprises cyclosporine, a hydrogenated polyoxyl castor oil, and octoxynol. In some embodiments, the hydrogenated polyoxyl castor oil is HCO-40. In some embodiments, the octoxynol is octoxynol-40. In some embodiments, the cyclosporine is about 0.087 to about 0.093% by weight of the formulation. In some embodiments, the cyclosporine is about 0.09% by weight of the formulation. In some embodiments, the formulation comprises about 0.09 wt % cyclosporine, about 0.5 wt % to about 2 wt % hydrogenated polyoxyl castor oil, e.g., HCO-40, and about 0.01 wt % to about 1 wt % octoxynol. In some embodiments, the formulation comprises about 0.09% wt cyclosporine, about 1.0 wt % HCO-40, and about 0.05 wt % octoxynol-40.

In some embodiments, the stable nanomicellar ophthalmic formulation provided herein is made by a method comprising:
a) mixing the polyoxyl lipid or fatty acid and the polyalkoxylated alcohol at a temperature of 100° C. or above, wherein the mixing produces a Mixture A comprising a water content of less than 3%;
b) adding the cyclosporine to the Mixture A to form a Mixture B; and
c) mixing the Mixture B with the aqueous vehicle, thereby forming the stable nanomicellar ophthalmic formulation.

In some embodiments, the concentrate comprising cyclosporine, the polyoxyl lipid or fatty acid, and the polyalkoxylated alcohol provided herein is made by a method comprising:
a) mixing the polyoxyl lipid or fatty acid and the polyalkoxylated alcohol at a temperatures of 100° C. or above, wherein the mixing produces a Mixture A comprising a water content of less than 3%;
b) adding the cyclosporine to the Mixture A to form a Mixture B, wherein the Mixture B comprises a water content of less than 3%.

In some embodiments, the disclosure provides a stable nanomicellar ophthalmic formulation comprising cyclosporine, hydrogenated 40 polyoxyl castor oil, octoxynol-40, and an aqueous vehicle, wherein the stable nanomicellar ophthalmic formulation is made by a method comprising:
a) mixing the hydrogenated 40 polyoxyl castor oil and the octoxynol-40 at a temperature of 100° C. or above, wherein the mixing produces a Mixture A comprising a water content of less than 3%;
b) adding the cyclosporine to the Mixture A to form a Mixture B; and
c) mixing the Mixture B with the aqueous vehicle, thereby forming the stable nanomicellar ophthalmic formulation.

In some embodiments, the disclosure provides a stable nanomicellar ophthalmic formulation comprising: i) a concentrate comprising cyclosporine, hydrogenated 40 polyoxyl castor oil, and octoxynol-40; and ii) an aqueous vehicle, wherein the concentrate is made by a method comprising:
a) mixing the hydrogenated 40 polyoxyl castor oil and the octoxynol-40 at a temperature of 100° C. or above, wherein the mixing produces a Mixture A comprising a water content of less than 3%; and
b) adding the cyclosporine to the Mixture A to form a Mixture B, wherein the Mixture B comprises a water content of less than 3%. In some embodiments, the formulation is made by mixing the concentrate with the aqueous vehicle.

In some embodiments, the disclosure provides a method of making a stable nanomicellar ophthalmic formulation, comprising:
a) mixing hydrogenated 40 polyoxyl castor oil and octoxynol-40 at a temperature of 100° C. or above, wherein the mixing produces a Mixture A comprising a water content of less than 3%;
b) adding cyclosporine to the Mixture A to form a Mixture B; and
c) mixing the Mixture B with the aqueous vehicle, thereby forming the stable nanomicellar ophthalmic formulation.

In some embodiments, the disclosure provides a method of making a stable nanomicellar ophthalmic formulation, comprising:
a) mixing hydrogenated 40 polyoxyl castor oil and octoxynol-40, wherein the octoxynol-40 comprises less than 3% impurities and a water content of less than 10%, wherein the mixing produces a Mixture A comprising a water content of less than 3%;
b) adding cyclosporine to the Mixture A to form a Mixture B; and
c) mixing the Mixture B with the aqueous vehicle, thereby forming the stable nanomicellar ophthalmic formulation.

In some embodiments, the disclosure provides a method of making a concentrate for a stable nanomicellar ophthalmic formulation, comprising:
a) mixing hydrogenated 40 polyoxyl castor oil and octoxynol-40 at a temperature of 100° C. or above, wherein the mixing produces a Mixture A comprising a water content of less than 3%; and
b) adding cyclosporine to the Mixture A to form a Mixture B, wherein the Mixture B comprises a water content of less than 3%.

In some embodiments, the disclosure provides a method of making a concentrate for a stable nanomicellar ophthalmic formulation, comprising:
a) mixing hydrogenated 40 polyoxyl castor oil and octoxynol-40, wherein the octoxynol-40 comprises less than 3% impurities and a water content of less than 10%, wherein the mixing produces a Mixture A comprising a water content of less than 3%; and
b) adding cyclosporine to the Mixture A to form a Mixture B, wherein the Mixture B comprises a water content of less than 3%.

It was surprisingly discovered that the methods provided herein, comprising mixing polyoxyl lipid or fatty acid, e.g., HCO-40, and polyalkoxylated alcohol, e.g., octoxynol-40, such that the resulting mixture (“Mixture A”) comprises a low water content, e.g., less than 3% water, followed by addition of cyclosporine, the formulation is highly stable even after long term storage. Further, any form of cyclosporine (e.g., any of Cs Forms 1, 2, or 3, or amorphous form described herein) can be used in the methods provided herein, which reduces complexity and cost of the manufacturing process. The methods also substantially reduce the interconversion of different forms of cyclosporine, thereby further improving stability of the formulation.

In some embodiments, the method comprises mixing hydrogenated 40 polyoxyl castor oil and octoxynol-40 to produce Mixture A. The mixing of hydrogenated 40 polyoxyl castor oil and octoxynol-40 to produce Mixture A is also referred to herein as “the mixing of a).” In some embodiments, the mixing of a) is performed for a sufficient amount of time and at sufficiently high temperature to substantially remove water from the hydrogenated 40 polyoxyl castor oil and/or the octoxynol-40.

In some embodiments, the octoxynol-40 that is mixed with the hydrogenated 40 polyoxyl castor oil is substantially free of impurities. In general, octoxynol-40 is produced from the reaction of 4-(1,1,3,3-tetramethylbutyl) phenol and 40 equivalents of ethyleneoxide in the presence of an acid or base catalyst, with residual ethylene oxide and the ethylene oxide cyclic dimer, 1,4-dioxane, as impurities. Compendium grade octoxynol-40 for use in pharmaceutical formulations is required to have less than 10 ppm (corresponding to 0.001%) ethylene oxide and less than 50 ppm (corresponding to 0.005%) 1,4-dioxane. Commercially available dry powder form of octoxynol-40 can contain up to about 40 ppm of ethylene oxide, which is far above the acceptable level for pharmaceutical use. Commercially available compendium grade octoxynol-40 is typically provided as a 70% aqueous solution.

In embodiments, the octoxynol-40 is substantially free of impurities. In some embodiments, the octoxynol-40 comprises less than 25 ppm total impurities, e.g., ethylene oxide and 1,4-dioxane. In some embodiments, the octoxynol-40 substantially free of impurities comprises a water content of at least 10%. In some embodiments, the octoxynol-40 comprises a water content of about 25% to about 35%. In some embodiments, the octoxynol-40 comprises a water content of about 30%. In such embodiments, the mixing of a) is performed at conditions sufficient to substantially remove the water from the octoxynol-40, e.g., at a temperature of 100° C. or above for at least 10 minutes.

In some embodiments, the mixing of a) is at a temperature of 100° C. or above, 110° C. or above, 120° C. or above, 130° C. or above, 140° C. or above, 150° C. or above, 160° C. or above, 170° C. or above, 180° C. or above, 190° C. or above, or 200° C. or above. In some embodiments, the mixing of a) is at a temperature of about 100° C. to about 200° C. In some embodiments, the mixing of a) is at a temperature of about 105° C. to about 180° C. In some embodiments, the mixing of a) is at a temperature of about 110° C. to about 160° C. In some embodiments, the mixing of a) is at a temperature of about 115° C. to about 150° C. In some embodiments, the mixing of a) is at a temperature of about 120° C. to about 140° C. In some embodiments, the mixing of a) is at a temperature of about 125° C. to about 135° C. In some embodiments, the mixing of a) is at a temperature of about 127° C. to about 130° C.

In some embodiments, the mixing of a) is performed for at least 5 minutes, at least 10 minutes, at least 15 minutes, at least 20 minutes, at least 30 minutes, at least 45 minutes, at least 60 minutes, at least 90 minutes, or at least 120 minutes. In some embodiments, the mixing of a) is performed for about 5 minutes to about 120 minutes. In some embodiments, the mixing of a) is performed for about 10 minutes to about 90 minutes. In some embodiments, the mixing of a) is performed for about 10 minutes to about 60 minutes. In some embodiments, the mixing of a) is performed for about 10 minutes to about 40 minutes. In some embodiments, the mixing of a) is performed for about 15 minutes to about 75 minutes. In some embodiments, the mixing of a) is performed for about 15 minutes to about 60 minutes. In some embodiments, the mixing of a) is performed for about 20 minutes to about 50 minutes. In some embodiments, the mixing of a) is performed for about 20 minutes to about 40 minutes. In some embodiments, the mixing of a) is performed for about 30 minutes to about 45 minutes.

In some embodiments, the mixing of a) is performed with a stirrer. In some embodiments, the mixing of a) is performed at about 50 to about 1000 rpm, or about 80 to about 900 rpm, or about 100 to about 800 rpm, or about 120 to about 700 rpm, or about 150 to about 600 rpm, or about 170 to about 500 rpm, or about 130 to about 400 rpm, or about 200 to about 400 rpm, or about 200 to about 300 rpm, or about 220 to about 380 rpm, or about 250 to about 350 rpm, or about 270 to about 320 rpm, or about 110 to about 200 rpm, or about 120 to about 190 rpm, or about 130 to about 180 rpm, or about 140 to about 170 rpm, or about 150 to about 160 rpm. In some embodiments, the mixing of a) is performed at 100 rpm or above, 110 rpm or above, 120 rpm or above, 130 rpm or above, 140 rpm or above, 150 rpm or above, 160 rpm or above, 170 rpm or above, 180 rpm or above, 190 rpm or above, or 200 rpm or above. In some embodiments, the mixing of a) is performed using a homogenizer, sonicator, or mixer comprising a stirrer. In some embodiments, the stirrer is located at the overhead and/or the bottom of the homogenizer, sonicator, or mixer.

In some embodiments, the mixing of a) is performed at any combination of time and temperature conditions according to Table 1.

TABLE 1

| Temperature | Time (min) | | | | | | | | | |
|---|---|---|---|---|---|---|---|---|---|---|
| ≥100 °C | ≥10 | ≥20 | 5-120 | 10-90 | 10-40 | 15-75 | 15-60 | 20-50 | 20-40 | 30-45 |
| ≥110 °C | ≥10 | ≥20 | 5-120 | 10-90 | 10-40 | 15-75 | 15-60 | 20-50 | 20-40 | 30-45 |
| ≥120 °C | ≥10 | ≥20 | 5-120 | 10-90 | 10-40 | 15-75 | 15-60 | 20-50 | 20-40 | 30-45 |
| ≥130 °C | ≥10 | ≥20 | 5-120 | 10-90 | 10-40 | 15-75 | 15-60 | 20-50 | 20-40 | 30-45 |
| ≥140 °C | ≥10 | ≥20 | 5-120 | 10-90 | 10-40 | 15-75 | 15-60 | 20-50 | 20-40 | 30-45 |
| ≥150 °C | ≥10 | ≥20 | 5-120 | 10-90 | 10-40 | 15-75 | 15-60 | 20-50 | 20-40 | 30-45 |
| 100-200 °C | ≥10 | ≥20 | 5-120 | 10-90 | 10-40 | 15-75 | 15-60 | 20-50 | 20-40 | 30-45 |
| 110-160 °C | ≥10 | ≥20 | 5-120 | 10-90 | 10-40 | 15-75 | 15-60 | 20-50 | 20-40 | 30-45 |
| 120-140 °C | ≥10 | ≥20 | 5-120 | 10-90 | 10-40 | 15-75 | 15-60 | 20-50 | 20-40 | 30-45 |
| 125-135 °C | ≥10 | ≥20 | 5-120 | 10-90 | 10-40 | 15-75 | 15-60 | 20-50 | 20-40 | 30-45 |
| 127-130 °C | ≥10 | ≥20 | 5-120 | 10-90 | 10-40 | 15-75 | 15-60 | 20-50 | 20-40 | 30-45 |

In some embodiments, the mixing of a) is performed at any combination of the time and temperature conditions according to Table 1, and at about 50 to about 1000 rpm. In some embodiments, the mixing of a) is performed at any combination of the time and temperature conditions according to Table 1, and at about 100 to about 800 rpm. In some embodiments, the mixing of a) is performed at any combination of the time and temperature conditions according to Table 1, and at about 120 to about 200 rpm. In some embodiments, the mixing of a) is performed at any combination of the time and temperature conditions according to Table 1, and at about 130 to about 180 rpm. In some embodiments, the mixing time and/or rpm required to provide a stable nanomicellar ophthalmic formulation is reduced relative to a method wherein the mixing of a) produces a Mixture A comprising a water content of greater than 3%.

In some embodiments, the time and temperature required in the mixing of a) is a function of the volume of Mixture A. In some embodiments, the mixing of a) is performed at any combination of the time and temperature conditions according to Table 1 when the method is performed in large quantities, e.g., wherein Mixture A is at least 10 L, at least 20 L, at least 30 L, at least 40 L, at least 50 L, at least 60 L, at least 70 L, at least 80 L, at least 90 L, at least 100 L, at least 200 L, at least 300 L, at least 400 L, at least 500 L, at least 600 L, at least 700 L, at least 800 L, at least 900 L, at least 1000 L, at least 1500 L, or at least 2000 L.

In some embodiments, the mixing of a) is performed at a temperature of 100° C. or above for at least 10 minutes at about 100 to about 800 rpm. In some embodiments, the mixing of a) is performed at a temperature of about 120° C. to about 140° C. for about 20 to about 60 minutes at about 130 to about 180 rpm.

In some embodiments, the octoxynol-40 that is mixed with the hydrogenated 40 polyoxyl castor oil is substantially free of impurities and has a water content of less than 10%, less than 5%, less than 3%, less than 2%, or less than 1%. In some embodiments, the octoxynol-40 comprises less than 25 ppm total impurities, e.g., ethylene oxide and 1,4-dioxane, and a water content of less than 10%. In some embodiments, the octoxynol-40 comprises less than 20 ppm, less than 15 ppm, less than 10 ppm, less than 5 ppm, less than 2 ppm, or less than 1 ppm total impurities and comprises a water content of less than 10%.

In some embodiments, the octoxynol-40 comprises total impurities at a level that is substantially undetectable, e.g., by gas chromatography (GC), differential scanning calorimetry (DSC), Thermo Gravimetric Analysis (TGA), or combination thereof, and comprises a water content of less than 10%. In some embodiments, the octoxynol-40 has a critical micellar concentration (CMC) of about 0.4 to about 0.7. In some embodiments, the octoxynol-40 has a CMC of about 0.5 to about 0.6.

In some embodiments, the octoxynol-40 is substantially free of impurities and comprises a water content of less than 10%, and the mixing of a) is performed under conditions sufficient to reduce the water content of the resulting Mixture A to less than 3%, or less than 2%, or less than 1%. In some embodiments, the octoxynol-40 is substantially free of impurities (e.g., less than 25 ppm, less than 20 ppm, less than 10 ppm, or less than 5 ppm total impurities as described herein) and comprises a water content of less than 10%, and the mixing of a) is at a temperature of 35° C. or above, 40° C. or above, 45° C. or above, 50° C. or above, 55° C. or above, 60° C. or above, 65° C. or above, 70° C. or above, 75° C. or above, 80° C. or above, 85° C. or above, 90° C. or above, 95° C. or above, or 100° C. or above.

In some embodiments, the octoxynol-40 comprises less than 25 ppm, less than 20 ppm, less than 10 ppm, or less than 5 ppm and a water content of less than 10%, and the mixing of a) is performed at any combination of time and temperature conditions according to Table 2.

TABLE 2

| Temperature | Time (min) | | | | | | | | | |
|---|---|---|---|---|---|---|---|---|---|---|
| ≥35 °C | ≥10 | ≥20 | 5-120 | 10-90 | 10-40 | 15-75 | 15-60 | 20-50 | 20-40 | 30-45 |
| ≥40 °C | ≥10 | ≥20 | 5-120 | 10-90 | 10-40 | 15-75 | 15-60 | 20-50 | 20-40 | 30-45 |
| ≥45 °C | ≥10 | ≥20 | 5-120 | 10-90 | 10-40 | 15-75 | 15-60 | 20-50 | 20-40 | 30-45 |
| ≥50 °C | ≥10 | ≥20 | 5-120 | 10-90 | 10-40 | 15-75 | 15-60 | 20-50 | 20-40 | 30-45 |
| ≥55 °C | ≥10 | ≥20 | 5-120 | 10-90 | 10-40 | 15-75 | 15-60 | 20-50 | 20-40 | 30-45 |
| ≥60 °C | ≥10 | ≥20 | 5-120 | 10-90 | 10-40 | 15-75 | 15-60 | 20-50 | 20-40 | 30-45 |
| ≥65 °C | ≥10 | ≥20 | 5-120 | 10-90 | 10-40 | 15-75 | 15-60 | 20-50 | 20-40 | 30-45 |
| ≥70 °C | ≥10 | ≥20 | 5-120 | 10-90 | 10-40 | 15-75 | 15-60 | 20-50 | 20-40 | 30-45 |
| ≥75 °C | ≥10 | ≥20 | 5-120 | 10-90 | 10-40 | 15-75 | 15-60 | 20-50 | 20-40 | 30-45 |
| ≥80 °C | ≥10 | ≥20 | 5-120 | 10-90 | 10-40 | 15-75 | 15-60 | 20-50 | 20-40 | 30-45 |
| ≥85 °C | ≥10 | ≥20 | 5-120 | 10-90 | 10-40 | 15-75 | 15-60 | 20-50 | 20-40 | 30-45 |
| ≥90 °C | ≥10 | ≥20 | 5-120 | 10-90 | 10-40 | 15-75 | 15-60 | 20-50 | 20-40 | 30-45 |
| ≥95 °C | ≥10 | ≥20 | 5-120 | 10-90 | 10-40 | 15-75 | 15-60 | 20-50 | 20-40 | 30-45 |
| ≥100 °C | ≥10 | ≥20 | 5-120 | 10-90 | 10-40 | 15-75 | 15-60 | 20-50 | 20-40 | 30-45 |

In some embodiments, the octoxynol-40 comprises less than 25 ppm, less than 20 ppm, less than 10 ppm, or less than 5 ppm total impurities and a water content of less than 10%, and the mixing of a) is performed at any combination of the time and temperature conditions according to Table 2, and at about 50 to about 1000 rpm. In some embodiments, the octoxynol-40 comprises less than 25 ppm, less than 20 ppm, less than 10 ppm, or less than 5 ppm total impurities and a water content of less than 10%, and the mixing of a) is performed at any combination of the time and temperature conditions according to Table 2, and at about 100 to about 800 rpm. In some embodiments, the octoxynol-40 comprises less than 25 ppm, less than 20 ppm, less than 10 ppm, or less than 5 ppm total impurities and a water content of less than 10%, and the mixing of a) is performed at any combination of the time and temperature conditions according to Table 2, and at about 120 to about 200 rpm. In some embodiments, the octoxynol-40 comprises less than 25 ppm, less than 20 ppm, less than 10 ppm, or less than 5 ppm total impurities and a water content of less than 10%, and the mixing of a) is performed at any combination of the time and temperature conditions according to Table 2, and at about 130 to about 180 rpm.

In some embodiments, the mixing of a) is performed at any combination of the time and temperature conditions according to Table 2 when the method is performed in large quantities, e.g., wherein Mixture A is at least 10 L, at least 20 L, at least 30 L, at least 40 L, at least 50 L, at least 60 L, at least 70 L, at least 80 L, at least 90 L, at least 100 L, at least 200 L, at least 300 L, at least 400 L, at least 500 L, at least 600 L, at least 700 L, at least 800 L, at least 900 L, at least 1000 L, at least 1500 L, or at least 2000 L.

In some embodiments, the octoxynol-40 comprises less than 25 ppm, less than 20 ppm, less than 10 ppm, or less than 5 ppm total impurities and a water content of less than 10%, and the mixing of a) is performed at a temperature of 55° C. or above for at least 10 minutes at about 100 to about 800 rpm. In some embodiments, the octoxynol-40 comprises less than 25 ppm, less than 20 ppm, less than 10 ppm, or less than 5 ppm total impurities and a water content of less than 10%, the mixing of a) is performed at a temperature of 100° C. or above for about 20 to about 60 minutes at about 130 to about 180 rpm.

In some embodiments, the Mixture A that is produced by the mixing of a) has a substantially low water content. In some embodiments, Mixture A comprises a water content of about 0.1% to about 3%, about 0.2% to about 2%, or about 0.5% to about 1%. In some embodiments, Mixture A comprises a water content of less than 3%, less than 2.5%, less than 2%, less than 1.5%, less than 1%, less than 0.5%, less than 0.4%, less than 0.3%, less than 0.2%, or less than 0.1%. In some embodiments, the low water content of Mixture A improves the stability of the cyclosporine added thereto. In some embodiments, the low water content of Mixture A reduces the interconversion between forms of the cyclosporine added thereto.

In some embodiments, the method comprises, following the mixing of a) as described herein, adding cyclosporine to the Mixture A to form a Mixture B. The method step comprising adding the cyclosporine to the Mixture A is also referred to herein as “step b).” The cyclosporine may be in any form described herein, e.g., (i) an amorphous form; (ii) a form with characteristic XRD peaks at 2-theta (deg.) 6.9, 7.8, 9.4 and 15.9 (i.e., Cs Form 1); (iii) a form with characteristic XRD peaks at 2-theta (deg.) 7.4, 8.7, 14.4 and 17.5 (i.e., Cs Form 2); (iv) a form with characteristic XRD peaks at 2-theta (deg.) 8.5, 9.3, 11.6 and 20.3 (i.e., Cs Form 3); or (v) any combination of (i)-(iv). In some embodiments, the cyclosporine is amorphous form. In some embodiments, the cyclosporine is Cs Form 1. In some embodiments, the cyclosporine is Cs Form 2. In some embodiments, the cyclosporine is Cs Form 3. In some embodiments, the cyclosporine comprises a combination of one or more of amorphous form, Cs Form 1, Cs Form 2, and Cs Form 3.

In some embodiments, the temperature of Mixture A is lowered prior to addition of the cyclosporine. In some embodiments, the cyclosporine is added to the Mixture A at a temperature of 35° C. or above, 40° C. or above, 45° C. or above, 50° C. or above, 55° C. or above, 60° C. or above, 65° C. or above, 70° C. or above, 75° C. or above, 80° C. or above, 85° C. or above, 90° C. or above, or 95° C. or above. In some embodiments, the cyclosporine is added to the Mixture A at a temperature of about 35° C. to about 75° C., or about 45° C. to about 65° C., or about 50° C. to about 60, or about 55° C.

In some embodiments, the temperature of Mixture A is not lowered prior to addition of the cyclosporine. In some embodiments, the cyclosporine is added to the Mixture A at the same temperature as the mixing of a). In some embodiments, the cyclosporine is added to the Mixture A at a temperature of 100° C. or above, 110° C. or above, 120° C. or above, 130° C. or above, 140° C. or above, 150° C. or above, 160° C. or above, 170° C. or above, 180° C. or above, 190° C. or above, or 200° C. or above. In some embodiments, the mixing is at a temperature of about 100° C. to about 200° C. In some embodiments, the mixing is at a temperature of about 105° C. to about 180° C. In some embodiments, the mixing is at a temperature of about 110° C. to about 160° C. In some embodiments, the mixing is at a temperature of about 115° C. to about 150° C. In some embodiments, the mixing is at a temperature of about 120° C. to about 140° C. In some embodiments, the mixing is at a temperature of about 125° C. to about 135° C. In some embodiments, the mixing is at a temperature of about 127° C. to about 130° C.

In some embodiments, the cyclosporine is added to the Mixture A and mixed for a sufficient period of time to completely dissolve the cyclosporine. The mixing of the cyclosporine with the Mixture A is also referred to herein as “the mixing of b).” The complete dissolution time of cyclosporine depends, e.g., on the quantity of cyclosporine to be dissolved and the batch size. In some embodiments, the mixing of b) is performed for at least 10 minutes, at least 15 minutes, at least 20 minutes, at least 30 minutes, at least 40 minutes, at least 45 minutes, at least 60 minutes, at least 75 minutes, at least 90 minutes, or at least 120 minutes. The mixing of b) may be performed by stirring, e.g., with a homogenizer, sonicator, or mixer comprising a stirrer as described herein.

In some embodiments, the temperature of the Mixture A is lowered prior to addition of the cyclosporine. In some embodiments, the mixing of a) is performed at 100° C. or above, and step b) is performed at 35° C. or above. In some embodiments, the mixing of a) is performed at 100° C. or above, and step b) is performed at 55° C. or above. In some embodiments, the mixing of a) and the mixing of b) are each performed for at least 10 minutes. In some embodiments, the mixing of a) and the mixing of b) are each performed for about 10 minutes to about 90 minutes. In some embodiments, the mixing of a) and the mixing of b) are each performed for about 20 minutes to about 60 minutes. In some embodiments, the mixing of a) is performed for at least 10 minutes, and the mixing of b) is performed until the cyclosporine is completely dissolved.

In some embodiments, the temperature of the Mixture A is not lowered prior to addition of the cyclosporine. In some embodiments, step b) is performed at the same temperature as the mixing of a). In some embodiments, the mixing of a) and step b) are both performed at 100° C. or above. In some embodiments, the mixing of a) and step b) are both performed at 110° C. or above. In some embodiments, the mixing of a) and step b) are both performed at 120° C. or above. In some embodiments, the mixing of a) and step b) are both performed at 130° C. or above. In some embodiments, the mixing of a) and step b) are both performed at about 100° C. to about 200° C. In some embodiments, the mixing of a) and step b) are both performed at about 110° C. to about 160° C. In some embodiments, the mixing of a) and step b) are both performed at about 120° C. to about 140° C. In some embodiments, the mixing of a) and step b) are both performed at about 125° C. to about 135° C. In some embodiments, the mixing of a) and step b) are both performed at about 127° C. to about 130° C. In some embodiments, the mixing of a) and the mixing of b) are each performed for at least 10 minutes. In some embodiments, the mixing of a) and the mixing of b) are each performed for about 10 minutes to about 90 minutes. In some embodiments, the mixing of a) and the mixing of b) are each performed for about 20 minutes to about 60 minutes. In some embodiments, the mixing of a) is performed for at least 10 minutes, and the mixing of b) is performed until the cyclosporine is completely dissolved.

In some embodiments, the Mixture B that is produced following step b) has a substantially low water content, e.g., the same or lower water content as the Mixture A described herein. In some embodiments, Mixture B comprises a water content of about 0.1% to about 3%, about 0.2% to about 2%, or about 0.5% to about 1%. In some embodiments, Mixture B comprises a water content of less than 3%, less than 2.5%, less than 2%, less than 1.5%, less than 1%, less than 0.5%, less than 0.4%, less than 0.3%, less than 0.2%, or less than 0.1%. In some embodiments, the low water content of Mixture B improves the stability of the nanomicellar ophthalmic formulation produced by the method. In some embodiments, the low water content of Mixture B reduces the interconversion between forms of cyclosporine in the nanomicellar ophthalmic formulation produced by the method.

In some embodiments, the result of step b) described herein is a concentrate that comprises hydrogenated 40 polyoxyl castor oil and octoxynol-40. In some embodiments, the concentrate is stored for a period of time prior to mixing with an aqueous vehicle to produce the stable nanomicellar ophthalmic formulation described herein. In some embodiments, the optimal storage conditions for the concentrate are simpler to maintain, e.g., closer to ambient temperature and humidity, as compared to the stable nanomicellar ophthalmic formulation. Thus, in some embodiments, it is advantageous to produce and store the concentrate until it is ready for final production, e.g., mixing with the aqueous vehicle to produce the stable nanomicellar ophthalmic formulation.

In some embodiments, the aqueous vehicle comprises sterile water. In some embodiments, the aqueous vehicle comprises water that is substantially free of contaminants. In some embodiments, the aqueous vehicle comprises water that is suitable for use in pharmaceutical formulations. In some embodiments, the aqueous vehicle comprises Water for Injection (WFI). WFI is sterile, nonpyrogenic, distilled water and typically contains less than 0.01 mg/mL of elements other than water.

In some embodiments, the formulation made by the method described herein comprises cyclosporine, hydrogenated 40 polyoxyl castor oil, and octoxynol-40, and further comprises one or more pharmaceutically acceptable excipients. Pharmaceutically acceptable excipients include but are not limited to additives, adjuvants, buffers, tonicity agents, bioadhesive polymers, pH adjusting agents, and preservatives. In some embodiments, the pharmaceutically acceptable excipient comprises a buffer, a tonicity agent, a bioadhesive agent, a pH adjusting agent, or combination thereof.

In some embodiments, the formulation provided herein comprises a buffer. Exemplary buffers include, without limitation, citrate buffer, phosphate buffer, Tris buffer, and borate buffer. In some embodiments, the buffer is phosphate buffer. In some embodiments, the phosphate buffer comprises sodium phosphate and/or potassium phosphate. In some embodiments, the phosphate buffer comprises sodium phosphate monobasic and/or sodium phosphate dibasic. In some embodiments, the buffer is about 0.3 to about 1.5 wt % of the formulation. In some embodiments, the buffer is about 0.4 to about 1.2 wt % of the formulation. In some embodiments, the buffer is about 0.45 to about 1.05 wt % of the formulation. In some embodiments, the buffer comprises a combination of sodium phosphate monobasic and sodium phosphate dibasic, wherein the sodium phosphate monobasic is about 0.15 to about 0.6 wt % of the formulation and the sodium phosphate dibasic is about 0.2 to about 0.5 wt % of the formulation. In some embodiments, the sodium phosphate monobasic is about 0.2 to about 0.55 wt % of the formulation, and the sodium phosphate dibasic is about 0.23 to about 0.465 wt % of the formulation.

In some embodiments, the formulation provided herein comprises a tonicity agent. Exemplary tonicity agents include, without limitation, mannitol, sodium chloride, sodium nitrate, sodium sulfate, dextrose, xylitol, sorbitol, trehalose, calcium chloride, and magnesium chloride. In some embodiments, the tonicity agent is sodium chloride. In some embodiments, the tonicity agent is about 0.01 to about 0.5 wt % of the formulation. In some embodiments, the tonicity agent is about 0.02 to about 0.2 wt % of the formulation. In some embodiments, the tonicity agent is about 0.03 to about 0.1 wt % of the formulation. In some embodiments, the tonicity agent is about 0.04 to about 0.06 wt % of the formulation. In some embodiments, the tonicity agent is about 0.05 wt % of the formulation. Tonicity agents may be used to adjust the osmolality of the compositions. In some embodiments, the osmolality of the formulation is about 100 to about 300 mOsmol/kg. In some embodiments, the osmolality of the formulation is about 150 to about 200 mOsmol/kg. In some embodiments, the osmolality of the formulation is about 160 to about 190 mOsmol/kg.

In some embodiments, the formulation provided herein comprises a bioadhesive agent. Bioadhesion refers to the ability of certain synthetic and biological macromolecules and hydrocolloids to adhere to biological tissues. Bioadhesive agents can enhance the viscosity of the formulation and thereby increase residence time in the eye. Exemplary bioadhesive polymers include, without limit, carboxylic polymers such as Carbopol® (carbomers); Noveon® (polycarbophils); cellulose derivatives including alkyl and hydroxyalkyl cellulose, such as methylcellulose, hydroxyethylcellulose (HEC), hydroxypropylcellulose (HPC), hydroxypropylmethylcellulose (HPMC), carboxymethylcellulose; gums such as locust bean, xanthan, agarose, karaya, and guar; and other polymers including but not limited to polyvinyl alcohol, povidone (also known as polyvinylpyrrolidone or PVP, including PVP K-30 and PVP K-90), polyethylene glycol, Pluronic® (Poloxamers), tragacanth, and hyaluronic acid; phase-transition polymers for providing sustained and controlled delivery of enclosed medicaments to the eye, e.g., alginic acid, carrageenans (e.g., Eucheuma), pectins, cellulose acetate phthalate, alkylhydroxyalkyl cellulose and derivatives thereof, hydroxyalkylated polyacrylic acids, and derivatives thereof, poloxamers and their derivatives, and the like. In some embodiments, the bioadhesive agent is a hydrophilic polymer selected from PVP, HPMC, HEC, polycarbophil, and combination thereof. In some embodiments, the bioadhesive agent is selected from PVP K-30, PVP K-90, and HPMC. In some embodiments, the bioadhesive agent is selected from PVP K-30 and PVP K-90. In some embodiments, the bioadhesive agent is about 0.05 to about 1 wt % of the formulation. In some embodiments, the bioadhesive agent is about 0.1 to about 0.7 wt % of the formulation. In some embodiments, the bioadhesive agent is about 0.2 to about 0.5 wt % of the formulation. In some embodiments, the bioadhesive agent is about 0.3 wt % of the formulation.

In some embodiments, the formulation provided herein further comprises a pH adjusting agent. In some embodiments, the pH adjusting agent comprises hydrochloric acid, sodium hydroxide, or combination thereof. In some embodiments, the pH of the formulation is about 5 to about 8. In some embodiments, the pH of the formulation is about 6 to about 7.5. In some embodiments, the pH of the formulation is about 6.5 to about 7.2.

In some embodiments, the formulation provided herein comprises an additive, e.g., a sugar, a glycerol, or a sugar alcohol. Pharmaceutical additives can be added to increase the efficacy or potency of other ingredients in the composition. For example, a pharmaceutical additive can be added to a formulation of the present disclosure to improve the stability of the cyclosporine, to adjust the osmolality of the composition, to adjust the viscosity of the composition, to facilitate drug delivery, or combination thereof. Non-limiting examples of pharmaceutical additives include a sugar such as, e.g., trehalose, mannose, D-galactose, lactose, or combination thereof.

In some embodiments, the formulation provided herein comprises a preservative. Exemplary preservatives include, but are not limited to, benzyl alcohol with or without EDTA, benzalkonium chloride, chlorhexidine, Cosmocil® CQ, or Dowicil® 200. In some embodiments, it may be desirable for a formulation as described herein to not include any preservatives. For example, in some embodiments, preservatives may not be necessary or desirable, e.g., when the formulation is included in single use containers. In some embodiments, it may be advantageous to include a preservative in the formulation, e.g., when the formulation is included in a multiuse container.

In some embodiments, the formulation provided herein comprises:
0.087 wt % to 0.093 wt % cyclosporine;
0.5 wt % to 2 wt % hydrogenated 40 polyoxyl castor oil; and
0.01 wt % to 1 wt % octoxynol-40.

In some embodiments, the formulation provided herein comprises:
0.09 wt % cyclosporine;
about 1.0 wt % hydrogenated 40 polyoxyl castor oil; and
about 0.05 wt % octoxynol-40.

In some embodiments, the cyclosporine in the formulation provided herein comprises any one or more of: (i) an amorphous form; (ii) a form with characteristic XRD peaks at 2-theta (deg.) 6.9, 7.8, 9.4 and 15.9 (Cs Form 1); (iii) a form with characteristic XRD peaks at 2-theta (deg.) 7.4, 8.7, 14.4 and 17.5 (Cs Form 2); (iv) a form with characteristic XRD peaks at 2-theta (deg.) 8.5, 9.3, 11.6 and 20.3 (Cs Form 3); or (v) any combination of (i)-(iv).

In some embodiments, the formulation provided herein comprises:
0.087 wt % to 0.093 wt % cyclosporine;
0.5 wt % to 2 wt % hydrogenated 40 polyoxyl castor oil; and
0.01 wt % to 1 wt % octoxynol-40;
0.01 wt % to 1 wt % sodium chloride;
0.1 wt % to 1.5 wt % povidone; phosphate buffer; and
sodium hydroxide and/or hydrochloric acid.

In some embodiments, the formulation provided herein comprises:
0.09 wt % cyclosporine;
about 1.0 wt % hydrogenated 40 polyoxyl castor oil;
about 0.05 wt % octoxynol-40;
about 0.05 wt % sodium chloride;
about 0.3 wt % povidone; phosphate buffer; and
sodium hydroxide and/or hydrochloric acid.

In some embodiments, the cyclosporine in the formulation provided herein comprises any one or more of: (i) an amorphous form; (ii) a form with characteristic XRD peaks at 2-theta (deg.) 6.9, 7.8, 9.4 and 15.9 (Cs Form 1); (iii) a form with characteristic XRD peaks at 2-theta (deg.) 7.4, 8.7, 14.4 and 17.5 (Cs Form 2); (iv) a form with characteristic XRD peaks at 2-theta (deg.) 8.5, 9.3, 11.6 and 20.3 (Cs Form 3); or (v) any combination of (i)-(iv).

In some embodiments, the phosphate buffer comprises sodium phosphate monobasic and/or sodium phosphate dibasic. In some embodiments, the phosphate buffer comprises 0.15 wt % to 0.6 wt % sodium phosphate monobasic; and 0.2 wt % to 0.5 wt % sodium phosphate dibasic. In some embodiments, the formulation comprises about 0.15 wt % to about 0.60 wt % sodium phosphate monobasic and about 0.17 wt % to about 0.56 wt % sodium phosphate dibasic. In some embodiments, the phosphate buffer comprises 0.2 wt % to 0.55 wt % sodium phosphate monobasic; and 0.23 wt % to 0.465 wt % sodium phosphate dibasic.

In some embodiments, the formulation provided herein comprises:
0.09 wt % cyclosporine;
about 1.0 wt % hydrogenated 40 polyoxyl castor oil;
about 0.05 wt % octoxynol-40;
about 0.05 wt % sodium chloride;
about 0.3 wt % povidone;
about 0.20-0.550 wt % sodium phosphate monobasic;
about 0.23-0.465 wt % sodium phosphate dibasic;
sodium hydroxide and/or hydrochloric acid; and
water, e.g., WFI,
wherein osmolality of the formulation is about 100 to about 300 mOsmol/kg, and
wherein pH of the formulation is about 5 to 8.

In some embodiments, the cyclosporine in the formulation provided herein comprises any one or more of: (i) an amorphous form; (ii) a form with characteristic XRD peaks at 2-theta (deg.) 6.9, 7.8, 9.4 and 15.9 (Cs Form 1); (iii) a form with characteristic XRD peaks at 2-theta (deg.) 7.4, 8.7, 14.4 and 17.5 (Cs Form 2); (iv) a form with characteristic XRD peaks at 2-theta (deg.) 8.5, 9.3, 11.6 and 20.3 (Cs Form 3); or (v) any combination of (i)-(iv).

In some embodiments, the formulation provided herein comprises:
0.09 wt % cyclosporine;
about 1.0 wt % hydrogenated 40 polyoxyl castor oil;
about 0.05 wt % octoxynol-40;
about 0.05 wt % sodium chloride;
about 0.3 wt % povidone;
about 0.20-0.550 wt % sodium phosphate monobasic;
about 0.23-0.465 wt % sodium phosphate dibasic;
sodium hydroxide and/or hydrochloric acid; and
water, e.g., WFI,
wherein osmolality of the formulation is about 150 to about 200 mOsmol/kg, and
wherein pH of the formulation is about 6.5 to 7.2.

In some embodiments, the cyclosporine in the formulation provided herein comprises any one or more of: (i) an amorphous form; (ii) a form with characteristic XRD peaks at 2-theta (deg.) 6.9, 7.8, 9.4 and 15.9 (Cs Form 1); (iii) a form with characteristic XRD peaks at 2-theta (deg.) 7.4, 8.7, 14.4 and 17.5 (Cs Form 2); (iv) a form with characteristic XRD peaks at 2-theta (deg.) 8.5, 9.3, 11.6 and 20.3 (Cs Form 3); or (v) any combination of (i)-(iv).

In some embodiments, the cyclosporine in the formulation is amorphous form. In some embodiments, the cyclosporine in the formulation is Cs Form 1. In some embodiments, the cyclosporine in the formulation is Cs Form 2. In some embodiments, the cyclosporine in the formulation is Cs Form 3. In some embodiments, the cyclosporine in the formulation comprises a combination of one or more of amorphous form, Cs Form 1, Cs Form 2, and Cs Form 3. In some embodiments, the cyclosporine in the formulation does not convert from one form to another.

In some embodiments, the formulation provided herein comprises a substantially low amount of an impurity characterized by a relative retention time (RRT) in a range of about 2.0 to about 2.7, wherein the RRT is determined by a method as described in the United States Pharmacopeia (USP) for cyclosporine (USP Revision Bulletin, Official Dec. 1, 2013). In some embodiments, the impurity is a degradation impurity of cyclosporine that may be formed during the manufacturing process of the formulation described herein. In some embodiments, the formulation comprises less than about 5 wt % of the said impurity. In some embodiments, the formulation comprises less than about 3 wt % of the said impurity. In some embodiments, the formulation comprises less than about 2 wt % of the said impurity. In some embodiments, the formulation comprises less than about 1 wt % of the said impurity. As used herein, “relative retention time (RRT)” is the retention time of a compound, e.g., cyclosporine or the impurity described herein, relative to the retention time of a reference standard as measured by HPLC. The reference standard may be, e.g., Cyclosporine USP reference standard. In some embodiments, the RRT of cyclosporine is about 0.9 to about 1.1, wherein the RRT is determined by a method as described in the United States Pharmacopeia (USP) for cyclosporine (USP Revision Bulletin, Official Dec. 1, 2013). In some embodiments, the formulation comprises less than about 2 wt % of an impurity characterized by a relative retention time (RRT) of about 2.0 to about 2.7.

The physico-chemical parameters of the ophthalmic nanomicellar ophthalmic formulation described herein include density, surface tension, and drop size (also known as droplet size). Extended structural characteristics of the formulation include, e.g., shape and morphology of nanomicelles in the formulation, which may be determined via Cryogenic Transmission Electronic Microscopy (Cryo TEM); particle size distribution of nanomicelles in the formulation; thermal characteristics, which may be determined via differential scanning calorimetry (DSC); and surface charge, which may be determined via dynamic light scattering (DLS).

In some embodiments, the density of the formulation provided herein is about 0.5 to about 2 g/ml. In some embodiments, the density of the formulation is about 0.8 to about 1.8 g/ml. In some embodiments, the density of the formulation is about 0.9 to about 1.2 g/ml. In some embodiments, the density of the formulation is about 1.0 to about 1.5 g/ml. In some embodiments, the density of the formulation is about 1.0 to about 1.1 g/ml. In some embodiments, the density of the formulation is about 1.001 to about 1.010 g/ml.

In some embodiments, the surface tension of the formulation provided herein is about 30 mN/m to about 40 mN/m. In some embodiments, the surface tension of the formulation is about 35 mN/m to about 40 mN/m. In some embodiments, the surface tension of the formulation is about 36 mN/m to about 39 mN/m. In some embodiments, the surface tension of the formulation is about 38 to about 39 mN/m. Surface tension measurement may be performed using a force tensiometer with a du Nouy ring, a Wilhelmy plate, or a platinum rod. In some embodiments, the surface tension of the formulation herein is measured using a platinum rod.

Drop size, also known as droplet size, refers to the volume of a product unit delivered in a single delivery, e.g., the volume of a single delivery of the formulation provided herein. In some embodiments, the drop size of the formulation is about 15 μL to about 40 μL. In some embodiments, the drop size of the formulation is about 18 μL to about 30 μL. In some embodiments, the drop size of the formulation is about 20 μL to about 40 μL. In some embodiments, the drop size of the formulation is about 22 μL to about 28 μL. In some embodiments, the drop size of the formulation is about 22 μL to about 25 μL.

In some embodiments, the formulation provided herein comprises mixed nanomicelles. In some embodiments, the average particle size (Z_{avg}) of the mixed nanomicelles is about 1-100 nm; or about 5-50 nm; or about 10-40 nm; or about 13-16 nm. In some embodiments, the Z_{avg} particle size of the mixed nanomicelles is about 10 to about 20 nm. In some embodiments, the Z_{avg} particle size of the mixed nanomicelles is about 12 to about 18 nm. In some embodiments, the Z_{avg} particle size of the mixed nanomicelles is about 13 to about 16 nm. In some embodiments, the Z_{avg} particle size of the mixed nanomicelles is about 14 to about 15 nm. In some embodiments, the polydispersity index (PDI) of the mixed nanomicelles is about 0.1 to about 0.5. In some embodiments, the PDI of the mixed nanomicelles is about 0.1 to about 0.3. Particle size and/or polydispersity index of nanomicelles may be measured, e.g., by laser light scattering or dynamic light scattering (DLS), e.g., with a Zetasizer, Malvern Instruments, New Jersey, USA.

Surface charge is the electric charge present at the surface of a particle in an aqueous solution, e.g., the mixed nanomicelles in the formulation provided herein, as affected by the pH and ionic strength of the aqueous solution. In general, mixed nanomicelles formed by non-ionic surfactants have a neutral charge (i.e., zeta potential of 0 mV). In some embodiments, the zeta potential of the formulation provided herein is about 0 mV=2 mV. In some embodiments, the zeta potential of the formulation provided herein is about 0 mV+1 mV. In some embodiments, the zeta potential of the formulation provided herein is about 0 mV+0.5 mV.

In some embodiments, the formulation provided herein has about 95% or higher cyclosporine encapsulation efficiency. In some embodiments, the formulation provided herein has about 96% or higher cyclosporine encapsulation efficiency. In some embodiments, the formulation provided herein has about 97% or greater cyclosporine encapsulation efficiency. In some embodiments, the formulation provided herein has about 98% or higher cyclosporine encapsulation efficiency. In some embodiments, the formulation provided herein has about 99% or higher cyclosporine encapsulation efficiency. In some embodiments, the formulation has about 100% cyclosporine encapsulation efficiency.

Cloud point of a formulation refers to the temperature at which precipitation occurs, resulting in turbidity or “cloudiness” in the formulation. In some embodiments, the cloud point of the formulation provided herein is about 40° C. to about 45° C. In some embodiments, the cloud point of the formulation is about 41° C. to about 44° C. In some embodiments, the cloud point of the formulation is about 42° C. to about 43° C.

In some embodiments, the formulation provided herein is made in a large scale batch, e.g., for pharmaceutical production. In some embodiments, the formulation is made in a batch size of at least 10 L, at least at least 20 L, at least 30 L, at least 40 L, at least 50 L, at least 60 L, at least 70 L, at least 80 L, at least 90 L, at least 100 L, at least 200 L, at least 300 L, at least 400 L, at least 500 L, at least 600 L, at least 700 L, at least 800 L, at least 900 L, at least 1000 L, at least 1500 L, or at least 2000 L. In some embodiments, the methods described herein are capable of being scaled to produce a large scale batch of the formulation. In some embodiments, the methods described herein make at least 10 L, at least 20 L, at least 30 L, at least 40 L, at least 50 L, at least 60 L, at least 70 L, at least 80 L, at least 90 L, at least 100 L, at least 200 L, at least 300 L, at least 400 L, at least 500 L, at least 600 L, at least 700 L, at least 800 L, at least 900 L, at least 1000 L, at least 1500 L, or at least 2000 L batch of the formulation.

In some embodiments, the formulation provided herein is stable at room temperature (about 20-25° C.) for about 6 to about 24 months. In some embodiments, the formulation is stable when maintained at room temperature for at least 6 months, at least 8 months, at least 10 months, at least 12 months, at least 14 months, at least 16 months, at least 18 months, at least 20 months, or at least 24 months.

In some embodiments, the disclosure provides a method of treating or preventing an ocular disease or condition, for example, dry eye, comprising administering a stable nanomicellar ophthalmic formulation made by the method described herein to a subject in need thereof. In some embodiments, the administering is at least 6 to 24 months after the formulation is made.

As used herein, the term “treating” refers to: preventing a disease, disorder or condition from occurring in a cell, a tissue, a system, animal or human which may be predisposed to the disease, disorder and/or condition but has not yet been diagnosed as having it; stabilizing a disease, disorder or condition, i.e., arresting its development; and/or relieving one or more symptoms of the disease, disorder or condition, i.e., causing regression of the disease, disorder and/or condition.

As used herein, a formulation that “prevents” a disorder or condition refers to a formulation that, in a statistical sample, reduces the occurrence of the disorder or condition in the treated sample relative to an untreated control sample, or delays the onset or reduces the severity of one or more symptoms of the disorder or condition relative to the untreated control sample.

As used herein, the term “ocular disease or condition” refers to diseases/conditions of the eye(s) that can be sight threatening, lead to eye discomfort, and may signal systemic health problems.

A subject to be treated by any of the formulations or methods of the present disclosure can be either a human or a non-human animal. In some embodiments, the present disclosure provides methods for the treatment of an ocular disease in a human subject in need thereof. In some embodiments, the present disclosure provides methods for the treatment of an inflammatory ocular disease in a human subject in need thereof. In some embodiments, the present disclosure provides methods for the treatment of an ocular disease in a veterinary subject in need thereof, including, but not limited to dogs, horses, cats, rabbits, gerbils, hamsters, rodents, birds, aquatic mammals, cattle, pigs, camelids, and other zoological animals.

In some embodiments, the formulation provided herein further comprises one or more additional active ingredients. In some embodiments, the one or more additional active ingredients comprises a resolvin, resolvin-like compound, a steroid, e.g., a corticosteroid, or combination thereof. In some embodiments, the one or more additional active ingredients comprises an antibiotic, e.g., azithromycin, ciprofloxacin, ofloxacin, gatifloxacin, levofloxacin, moxifloxacin, besifloxacin, levofloxacin, or combination thereof. In some embodiments, the one or more additional active ingredients comprises an antibiotic described herein and a resolvin. In some embodiments, the one or more additional active ingredients comprises an antiviral, e.g., ganciclovir, trifluridine, acyclovir, famciclovir, valacyclovir, penciclovir, cidofovir, or combination thereof. In some embodiments, the one or more additional active ingredients comprises an antiviral described herein and a resolvin.

In some embodiments, the ocular disease is an anterior segment disease. In some embodiments, the ocular disease is a posterior segment disease. In some embodiments, the ocular disease is selected from dry eye syndrome, Sjogren's syndrome, uveitis, anterior uveitis (iritis), chorioretinitis, posterior uveitis, conjunctivitis, allergic conjunctivitis, keratitis, keratoconjunctivitis, vernal keratoconjunctivitis (VKC), atopic keratoconjunctivitis, systemic immune mediated diseases such as cicatrizing conjunctivitis and other autoimmune disorders of the ocular surface, blepharitis, scleritis, age-related macular degeneration (AMD), diabetic retinopathy (DR), diabetic macular edema (DME), ocular neovascularization, age-related macular degeneration (ARMD), proliferative vitreoretinopathy (PVR), cytomegalovirus (CMV) retinitis, optic neuritis, retrobulbar neuritis, and macular pucker. In some embodiments, the ocular disease is dry eye. In some embodiments, the ocular disease is allergic conjunctivitis. In some embodiments, the ocular disease is age-related macular degeneration (AMD). In some embodiments, the ocular disease is diabetic retinopathy.

In some embodiments, the formulation is administered as a single daily dose or several unit doses. In some embodiments, the formulation is administered once daily. In some embodiments, the formulation is administered twice daily. In some embodiments, the formulation is administered on an as-needed basis for the subject. In some embodiments, each dose comprises twice daily administration (approximately 12 hours apart) of one drop of the formulation comprising 0.09 wt % cyclosporine per eye.

### EXAMPLES

### Example 1. Manufacturing Method

Polyoxyl 40 hydrogenated castor oil (Kolliphor® RH 40) was heated to about 50-60° C., until liquefied prior to introduction into a 10 L glass vessel. The temperature was increased to 127-130° C. Octoxynol-40 was then added. If the octoxynol-40 solidified, it was heated at about 50-60° C. until it liquefies prior to its addition. The mixture was stirred for 20 minutes at 127-130° C. Cyclosporine (CsA) was added while maintaining the vessel temperature at 127-130° C. and stirred at approximately 200-300 RPM using a stirrer for complete dissolution.

A portion (approximately 90%) of Water for Injection (WFI) was charged into the stainless-steel mixing tank and the temperature was maintained at 20-30° C. throughout the process. While stirring, the CsA mixture was added at 127-130° C. to the mixing tank and stirred for approximately 15 minutes using both an overhead and bottom stirrer while the remaining excipients are added in the following order: sodium phosphate monobasic, sodium phosphate dibasic, sodium chloride, and polyvinylpyrrolidone. [0105] After mixing for 15 minutes, the pH was checked and adjusted, if necessary, to 6.8±0.2 using hydrochloric acid (IN) and/or sodium hydroxide (IN). The solution was adjusted to the final volume with WFI and filtered through a 0.2 μm filter.

The final formulation includes:

| Ingredient | Amount | |
|---|---|---|
| Cyclosporine | 0.09 | wt % |
| hydrogenated 40 polyoxyl castor oil, | 1.0 | wt % |
| octoxynol-40 | 0.05 | wt % |
| sodium phosphate monobasic | 0.53 | wt % |
| sodium phosphate dibasic | 0.47 | wt % |
| sodium chloride | 0.05 | wt % |
| povidone | 0.3 | wt % |

| | |
|---|---|
| sodium hydroxide | q.s. to adjust pH, if required |
| hydrochloric acid | q.s. to adjust pH, if required |
| water for injection | q.s. to 100% |

### Example 2. Water Content Measurement

Theoretical and actual water content of formulations prepared by methods described herein were determined.

### Theoretical Water Content:

A. HCO-40 with 2% Water Content.

Water limit specification of HCO-40 is no more than 2% as per the Vendor COA. The solid content specification of octoxynol-40 (OC-40) is 67-73%; thus, water content of OC-40 is 33-27%. A 100 mL batch contains 1% HCO-40 and 0.05% OC-40.
Non aqueous phase for 100 ml formulation 1+0.05=1.05 μm Content of water from 1 μm HCO-40 (2% per product spec)=0.020 μm
Content of water from 0.05 μm OC-40 (33% per product spec)=0.017 μm
Total water content=0.020+0.017=0.037 μm in 1.05 μm of non-aqueous phase
Theoretical % of water content=3.5%
   B. HCO-40 with 3% Water Content.
Water limit specification of HCO-40 is no more than 3% as per USP and followed by the drug product manufacturing site.
Total water content=0.030+0.017=0.047 μm in 1.05 μm of non-aqueous phase
Theoretical maximum % of water content theoretically=4.48%

### C. Lower Limit of OC-40 Water Content (27%).

Total water content=0.020+0.014=0.034 μm in 1.05 μm of non-aqueous phase
Theoretical % of water content=3.2%
   D. HCO-40 Lot a with 1% Water Content.
Total water content=0.010+0.014=0.024 μm in 1.05 μm of non-aqueous phase
Theoretical % of water content=2.28%
   E. HCO-40 Lot B with 0.6% Water Content.
Total water content=0.006+0.014=0.02 μm in 1.05 μm of non-aqueous phase
Theoretical minimum % of water content=1.90%

### Actual Water Content Measured:

Method I. Accurately weighed 10 μm of pre-heated (55±2° C.) HCO-40 was taken into a glass beaker. 0.5 μm OC-40 was added into the glass beaker. The solution was mixed at 55±2° C. for 10 min. The material was collected into the sampling vial, sealed properly and sent for analysis.
Water content result: 2.025%.

Method II. Accurately weighed 10 μm of pre-heated (55±2° C.) HCO-40 was taken into a glass beaker. 0.5 μm OC-40 was added into the glass beaker. Temperature was increased to 127±2° C. Mix the solution at 127±2° C. for 20 min. The material was collected into the sampling vial, sealed properly and sent for analysis.
Water content result: 0.205%.

### Example 3. Observation of Impurity

During the dissolution of cyclosporine in the mixture of HCO-40 and OC-40 at 127±2° C., the isomeric impurity with an RRT (relative retention time) value between 2.0 to 2.7 was observed increasingly with time. Results are shown in Table 3.

TABLE 3

| Heat | API @ 127 °C delivery | | | | | |
|---|---|---|---|---|---|---|
| exposure time | 10 min | 15 min | 18 min | 20 min | 30 min | 40 min |
| Test | Batch | Batch | Batch | Batch | Batch | Batch |
| | 1 | 2 | 3 | 4 | 5 | 6 |
| Impurity | 0.562% | 0.696% | 0.797% | 0.91% | 0.957% | 1.226 |
| (RRT of | | | | | | |
| 2.0 to 2.7) | | | | | | |

The impurity was determined by HPLC using the method described in the United States Pharmacopeia (USP) for cyclosporine (USP Revision Bulletin, Official Dec. 1, 2013). Briefly, the HPLC was performed as follows with a Hypersil™ ODS 250 mm×4.6 mm, 3 μm column. Detector was set at 210 nm. Column temperature was maintained at 80-80° C. Flow rate of mobile phase was 1.3-1.7 mL/min. Mobile phase included acetonitrile, sodium dodecyl sulfate, phosphoric acid, tert-butylmethyl ether (anhydrous), and purified water having a pH range of 2-4 (adjusted with sodium hydroxide). The total run time was 40 minutes for standards and 90 minutes for blanks, placebo, and sample solutions.

### Example 4. Purification of Octoxynol-40

Commercial grade octoxynol-40 can contain residual quantities of 1,4-dioxane and ethylene oxide as described herein. A purification process to reduce the levels of these impurities to acceptable levels was undertaken. The purification process involves an azeotropic, vacuum distillation at temperature ≤60° C., after dilution with water for injection. Octoxynol-40 is supplied as an aqueous solution containing 70% solids. Pre-dilution with water for injection assures that the solution after distillation is the same concentration as supplied by the vendor.

The final concentration of solids in the purified Octoxynol-40 is measured as an in-process control, determined by ultraviolet spectroscopy, prior to dispensing from the reactor into the final packaging container which ensures this critical excipient maintains the functionality to support. Release testing results and stability data from 3 lots at 25° C./60% RH and 40° C./75% RH indicate that the purification process does not adversely change the molecular weight distribution of the surfactant nor does it change its critical micellar concentration (CMC), which is indicative that the surface active functionality of this material, which is required to form the final drug product formulation, has not changed.

### Example 5. Evaluation of the Physico-Chemical Characteristics of the Composition

The physico-chemical parameters of the composition prepared from the manufacturing process of Example 1 were determined. The physico-chemical parameters that were evaluated for 3 batches of the composition prepared according to the process of Example 1 (hereinafter referred to as “Batches 1-3”) include: density, surface tension, drop size, and extended structural characterization of the obtained micelles via Cryogenic Transmission Electronic Microscopy (Cryo TEM), particle size distribution, Differential Scanning calorimetry (DSC), and surface charge measurement.

### A. Density.

Density of Batches 1-3 were measured, and results are provided in Table 4.

TABLE 4
Density Results

| Sample | Density (gm/ml) |
|---|---|
| Batch 1 | 1.007 |
| Batch 2 | 1.007 |
| Batch 3 | 1.007 |

### B. Surface Tension.

Surface tension of Batches 1-3 were measured using a direct surface technique utilizing a platinum rod, and results are shown in Table 5.

TABLE 5
Surface Tension Results

| Sample | Surface Tension |
|---|---|
| Batch 1 | 38.77 |
| Batch 2 | 38.35 |
| Batch 3 | 38.55 |

### C. Drop Size

A drop size dose delivery performance test was conducted to determine the average drop size of Batches 1-3, and results are shown in Table 6.

TABLE 6
Drop Size Results

| Sample | Average drop size (μL) |
|---|---|
| Batch 1 | 23.4 |
| Batch 2 | 23.7 |
| Batch 3 | 23.4 |

### D. Shapes and Surface Morphology

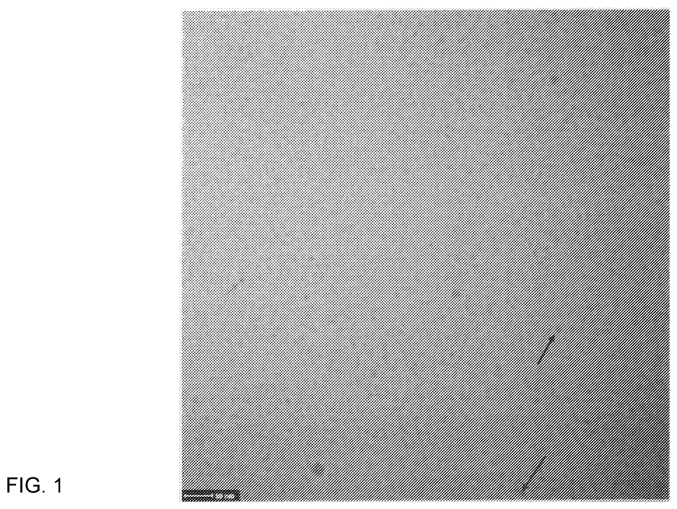

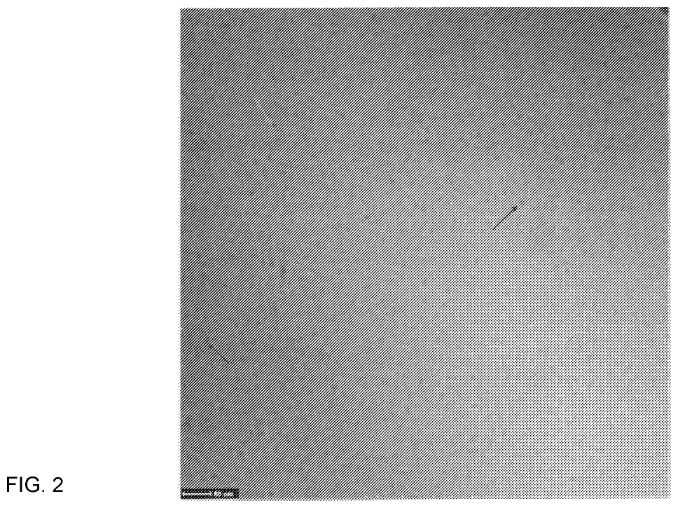

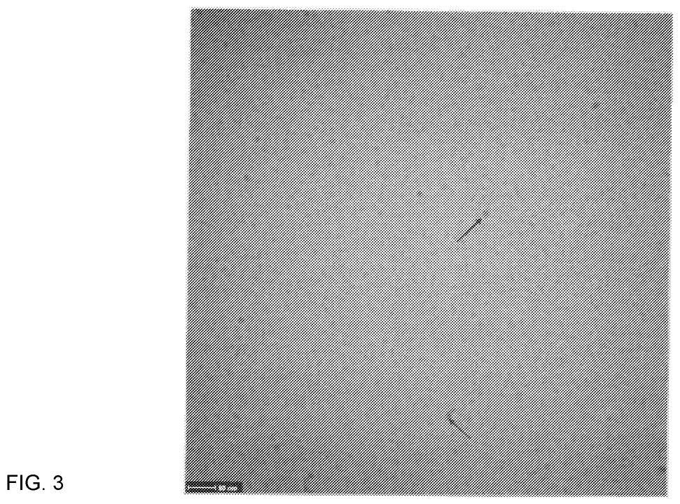

Cryo TEM was used to determine the shapes and surface morphology of Batches 1-3. The size distribution was visualized at a scale bar of 50 nm. The Cryo TEM images are shown in FIGS. 1-3 and appeared to show many small, slightly dense particles of 8-12 nm in diameter, a few small particles 2-4 nm in diameter, and very few isolated particles 20-30 micron in diameter. The particles were homogeneous and well dispersed, and no aggregation was observed (FIGS. 1, 2, and 3, corresponding to Batches 1, 2, and 3, respectively).

### E. Particle Size

The particle size of nanomicelles in Batches 1-3 were measured by laser light scattering, and results are summarized in Table 7.

TABLE 7
Particle Size Measurements

| Sample | Particle Size |
|---|---|
| Batch-1 | Z_{avg} = 15 (nm) PDI = 0.14 |
| Batch-2 | Z_{avg} = 14 (nm) PDI = 0.13 |
| Batch-3 | Z_{avg} = 15 (nm) PDI = 0.2 |

### F. Thermal Characteristics

Batches 1-3 were evaluated by a Nano Differential Scanning calorimeter (TA Instruments) for their thermal characteristics. The results of the scans illustrated thermal events that occurred during the process as the temperature was increased.

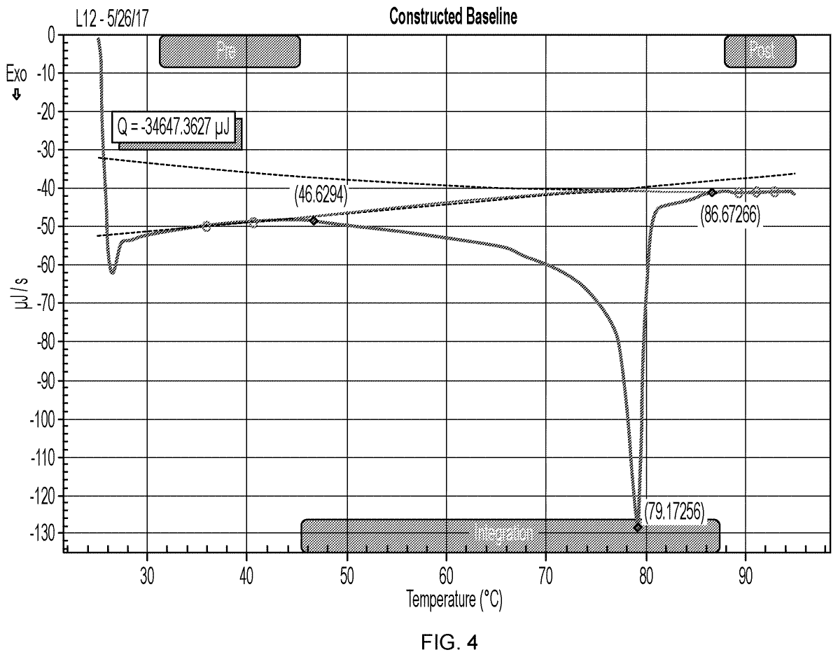

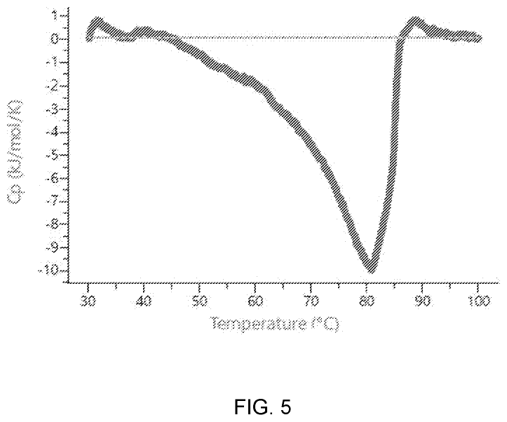

An early event started to occur at around 46° C. and was interpreted as the onset of micelle dissociation. A second peak was observed at above 80° C., which was interpreted as the peak of the demicellization event (see FIG. 4, FIG. 5, and Table 8).

The total heat evolved in the placebo and Batches 1-3 were measured from the area under the power/temperature plot, and greater heat evolution was observed in clinical batches in comparison to placebo.

This greater heat liberation was due to the presence of cyclosporine in the micelles. The phenomenon was observed and evaluated by MicroCal PEAQ-DSC Automated (Malvern Instruments).

TABLE 8
Average Quantitative DSC Temperature Results
from Old and New Process Batches

| | Onset of | Peak of Exotherm | |
|---|---|---|---|
| Sample | Demicellization (°C) | (°C) | Heat (mJ) |
| Batch-1 | 44.29 | 86.65 | −14.1 |
| Batch-2 | 46.18 | 86.85 | −15.2 |
| Batch-3 | 43.53 | 88.60 | −13.6 |

### G. Surface Charges

Surface charges of the nanomicelles of Batches 1-3 were determined using Dynamic Light Scattering (DLS), and the zeta potential results are provided in Table 9. These results illustrate that the zeta potential is about 0 mV (within experimental variability) for Batch 1-3. Without being bound by theory, it is believed that the absence of charge or the neutral charge is due to the fact that the nanomicelles are formed from non-ionic surfactant in the composition.

TABLE 9
Zeta Potential Results

Zeta Potential (mV)

| Sample | Average | Std Dev |
|---|---|---|
| Batch-1 | −0.7 | 0.4 |
| Batch-2 | −1.6 | 0.2 |
| Batch-3 | −0.4 | 1.1 |

### Example 6

To determine the efficacy and safety of the cyclosporine-containing compositions, encapsulation efficiency, cloud point and relaxation time were determined for Batches 1-3.

### A. Free Drug Content and Encapsulation Efficiency

Samples from Batches 1-3 were passed through 30 kDa 0.5 mL centrifugal filter and analyzed for free cyclosporine. Using the assay value of the batches and respective free drug content, encapsulation efficiency was calculated. Table 11 shows the comparative free drug content and encapsulation efficiency of Batches 1-3. The results demonstrated that about 100% of the cyclosporine is entrapped within the micelles.

TABLE 11
Encapsulation Efficiency and Free Drug

| | Encapsulation | |
|---|---|---|
| Sample | Efficiency (%) | Free Drug (%) |
| Batch-1 | 99.9 | 0.2 |
| Batch-2 | 99.8 | 0.2 |
| Batch-3 | 99.9 | 0.1 |

### B. Cloud Point/Relaxation Time

The cloud point and relaxation time of Batches 1-3 were measured. The sample was placed in a glass test tube and was heated in a heating block in 5° C. increments, noting the temperature when the sample becomes cloudy. This temperature is the Cloud Point of the sample.

Immediately after the sample was heated past the cloud point, it was transferred into a UV-Visible cuvette to begin recording the absorbance at 400 nm. Absorbance was recorded every 30 seconds until the spectrum stopped changing. The time for this process was recorded as the Relaxation Time.

Results are shown in Table 12.

TABLE 12
Cloud Points and Relaxation Time Results

| Sample | Cloud point (°C) | Relaxation Time |
|---|---|---|
| Batch-1 | 43 | 11 min |
| Batch-2 | 42.9 | 11 min |
| Batch-3 | 43 | 11 min |

### Example 7

Samples of the composition prepared according to the process of Example 1 were kept for stability study under stress conditions of 40° C./75% RH and 25° C./75% RH. The study results are shown in Tables 13 and 14.

TABLE 13
Stability Testing Result at 25 °C/40% RH
Stability Testing Result under 25 °C/40% RH

| | | | Time Points | | | | |
|---|---|---|---|---|---|---|---|
| Parameters | Analytical Method Version | | | 3 | 6 | 9 | 12 |
| Tests | Specifications | | Initial | months | months | months | months |
| Appearance | Clear colorless solution, | | Complies | Complies | Complies | Complies | Complies |
| | essentially free from visible | | | | | | |
| | particulate matter | | | | | | |
| pH | 6.5-7.2 | | 6.8 | 6.8 | 6.8 | 6.8 | 6.8 |
| Osmolality | 160-190 mOsmol/kg | | 166 | 169 | 171 | 171 | 171 |
| Viscosity | 1.3-2.0 cP | | 1.5 | 1.5 | 1.6 | 1.4 | 1.4 |
| HPLC method | | | | | | | |
| II: | | | | | | | |
| Cyclosporine | 90.0-105.0% of label claim | | 97.1 | 98.6 | 97.9 | 99.9 | 98.5 |
| Assay | | | | | | | |
| Specified | ≤0.7% | Dihydrocyclosporine A + | 0.27 | 0.27 | 0.22 | 0.27 | 0.23 |
| impurities | | Cyclosporine V | | | | | |
| (identified and | | Cyclosporine D | 0.10 | 0.11 | 0.09 | 0.10 | 0.10 |
| unidentified) | | RRT of 2.0 to 2.7 | 0.46 | 0.22 | 0.24 | 0.20 | 0.23 |
| | | RRT 0.36 | <0.05 | <0.05 | 0.06 | 0.17 | ND |
| | | RRT 0.55 | <0.05 | <0.05 | <0.05 | ND | ND |
| | | Cyclosporine H | ND | ND | ND | ND | ND |
| Unspecified | ≤0.1% | RRT 0.78 | 0.06 | <0.05 | ND | <0.05 | <0.05 |
| impurities | | RRT 1.29 | 0.07 | 0.07 | ND | 0.05 | 0.05 |
| | | RRT: | — | | | | |
| | | RRT: | — | — | | | |

| | | | | | | |
|---|---|---|---|---|---|---|
| Total | ≤2.0% | 0.95 | 0.68 | 0.61 | 0.80 | 0.61 |
| impurities | | | | | | |
| Micelle size | Z_{avg}: 10-20 nm | 15 | 14 | 14 | 14 | 15 |
| determination | PDI ≤0.2 | 0.14 | 0.13 | 0.13 | 0.12 | 0.13 |

TABLE 14
Stability Testing Result under 40 °C/NMT25% RH
Stability Testing Result under 40 °C/NMT25% RH

| | Analytical Method Version | | Time Points | | | |
|---|---|---|---|---|---|---|
| Tests | Specifications | | Initial | 1 month | 3 months | 6 months |
| Appearance | Clear colorless solution essentially free | | Complies | Complies | Complies | Co lies |
| | from visible particulate matter | | | | | |
| pH | 6.5-7.2 | | 6.8 | 6.8 | 6.8 | 68 |
| Osmolality | 160-190 mOsmol/kg | | 166 | 165 | 172 | 171 |
| Viscosity | 1.3-2.0 cP | | 15 | 1.5 | 1.5 | 1.4 |
| HPLC method II: | | | | | | |
| Cyclosporine | 90.0-105.0% of label claim | | 97.1 | 90.3 | 93.8 | 93.1 |
| Assay | | | | | | |
| Specified | ≤0.7% | Dihydrocyclosporine A + | 0.27 | 0.20 | 0.27 | 0.17 |
| impurities | | Cyclosporine V | | | | |
| (identified and | | Cyclosporine D | 0.10 | 0.10 | 0.11 | 0.09 |
| unidentified) | | RRT-2.0-2.7 | 0.46 | 0.13 | 0.34 | 0.17 |
| | | RRT0.36 | <0.05 | <0.05 | 0.08 | 0.13 |
| | | RRT0.55 | <0.05 | <0.05 | <0.05 | ND |
| | | Cyclosporine H | ND | ND | ND | ND |
| Unspecified | ≤0.1% | RRT 0.78 | 0.06 | <0.05 | <0.05 | ND |
| impurities | | RRT 1.29 | 0.07 | <0.05 | 0.06 | ND |
| | | RRT: | | — | — | |
| | | RRT: | | — | — | — |

| | | | | | |
|---|---|---|---|---|---|
| Total impurities | ≤2.0% | 0.95 | 0.42 | 0.85 | 0.56 |
| Micelle size | Z_{avg}: 10-20 nm | 15 | 15 | 14 | 14 |
| determination | PDI ≤0.2 | 0.14 | 0.17 | 0.13 | 0.13 |

Samples of test formulation were found to be stable and within specification throughout the stability testing period. The samples were found stable for at least 6 months at 40° C./75% RH and at least 12 months at 25° C./40% RH.

1. A stable nanomicellar ophthalmic formulation comprising cyclosporine, hydrogenated 40 polyoxyl castor oil, octoxynol-40, and an aqueous vehicle, wherein the stable nanomicellar ophthalmic formulation is made by a method comprising:
a) mixing the hydrogenated 40 polyoxyl castor oil and the octoxynol-40 at a temperature of 100° C. or above, wherein the mixing produces a Mixture A comprising a water content of less than 3%;
b) adding the cyclosporine to the Mixture A to form a Mixture B; and
c) mixing the Mixture B with the aqueous vehicle, thereby forming the stable nanomicellar ophthalmic formulation.

2. The stable nanomicellar ophthalmic formulation of claim 1, wherein the Mixture B comprises a water content below about 3%.

3. The stable nanomicellar ophthalmic formulation of claim 1 or 2, wherein the water content of Mixture A and/or the water content of Mixture B is below about 2%.

4. The stable nanomicellar ophthalmic formulation of any one of claims 1 to 3, wherein the water content of Mixture A and/or the water content of Mixture B is below about 1%.

5. A stable nanomicellar ophthalmic formulation comprising:
i) a concentrate comprising cyclosporine, hydrogenated 40 polyoxyl castor oil, and octoxynol-40; and
ii) an aqueous vehicle,
wherein the concentrate is made by a method comprising:
a) mixing the hydrogenated 40 polyoxyl castor oil and the octoxynol-40 at a temperature of 100° C. or above, wherein the mixing produces a Mixture A comprising a water content of less than 3%;
b) adding the cyclosporine to the Mixture A to form a Mixture B, wherein the Mixture B comprises a water content of less than 3%.

6. The stable nanomicellar ophthalmic formulation of claim 5, wherein the formulation is made by mixing the concentrate with the aqueous vehicle.

7. The stable nanomicellar ophthalmic formulation of any one of claims 1 to 6, wherein the water content of Mixture A and/or the water content of Mixture B is below about 0.5%.

8. The stable nanomicellar ophthalmic formulation of any one of claims 1 to 7, wherein the mixing of a) is at a temperature of about 100° C. to about 200° C.

9. The stable nanomicellar ophthalmic formulation of any one of claims 1 to 8, wherein the mixing of a) is at a temperature of about 110° C. to about 160° C.

10. The stable nanomicellar ophthalmic formulation of any one of claims 1 to 9, wherein the mixing of a) is at a temperature of about 120° C. to about 140° C.

11. The stable nanomicellar ophthalmic formulation of any one of claims 1 to 10, wherein the mixing of a) is at a temperature of about 127° C. to about 130° C.

12. The stable nanomicellar ophthalmic formulation of any one of claims 1 to 7, wherein the mixing of a) is at a temperature of about 130° C. or above.

13. The stable nanomicellar ophthalmic formulation of any one of claims 1 to 12, wherein the mixing of a) is performed for about 10 minutes to about 90 minutes.

14. The stable nanomicellar ophthalmic formulation of any one of claims 1 to 13, wherein the mixing of a) is performed for about 15 minutes to about 60 minutes.

15. The stable nanomicellar ophthalmic formulation of any one of claims 1 to 14, wherein the mixing of a) is performed for about 20 minutes to about 40 minutes.

16. The stable nanomicellar ophthalmic formulation of any one of claims 1 to 15, wherein the mixing of a) is performed at about 100 to about 800 rpm.

17. The stable nanomicellar ophthalmic formulation of any one of claims 1 to 16, wherein the mixing of a) is performed at about 130 to about 180 rpm.

18. The stable nanomicellar ophthalmic formulation of any one of claims 1 to 17, wherein the octoxynol-40 comprises less than 20 ppm total impurities.

19. The stable nanomicellar ophthalmic formulation of any one of claims 1 to 18, wherein the octoxynol-40 comprises less than 10 ppm total impurities.

20. The stable nanomicellar ophthalmic formulation of any one of claims 1 to 19, wherein the octoxynol-40 comprises less than 5 ppm total impurities.

21. The stable nanomicellar ophthalmic formulation of any one of claims 1 to 20, wherein step b) is performed at a temperature of 55° C. or above.

22. The stable nanomicellar ophthalmic formulation of any one of claims 1 to 21, wherein step b) is performed at a temperature of 100° C. or above.

23. The stable nanomicellar ophthalmic formulation of any one of claims 1 to 22, wherein step b) is performed at a temperature of 130° C. or above.

24. The stable nanomicellar ophthalmic formulation of any one of claims 1 to 20, wherein step b) is performed at the same temperature as the mixing of a).

25. The stable nanomicellar ophthalmic formulation of any one of claims 1 to 24, wherein step b) further comprises mixing until the cyclosporine is completely dissolved.

26. The stable nanomicellar ophthalmic formulation of any one of claims 1 to 25, wherein the aqueous vehicle comprises Water for Injection (WFI).

27. The stable nanomicellar ophthalmic formulation of any one of claims 1 to 26, further comprising a buffer, a tonicity agent, a bioadhesive agent, a pH adjusting agent, or combination thereof.

28. The stable nanomicellar ophthalmic formulation of claim 27, wherein the buffer comprises phosphate buffer; the tonicity agent comprises sodium chloride, the bioadhesive agent comprises povidone; and the pH adjusting agent comprises sodium hydroxide and/or hydrochloric acid.

29. The stable nanomicellar ophthalmic formulation of any one of claims 1 to 28, comprising:
0.09 wt % cyclosporine;
about 1.0 wt % hydrogenated 40 polyoxyl castor oil; and
about 0.05 wt % octoxynol-40.

30. The stable nanomicellar ophthalmic formulation of any one of claims 1 to 29, comprising:
0.09 wt % cyclosporine;
about 1.0 wt % hydrogenated 40 polyoxyl castor oil; about 0.05 wt % octoxynol-40;
about 0.05 wt % sodium chloride; about 0.3 wt % povidone;
phosphate buffer; and
sodium hydroxide and/or hydrochloric acid.

31. The stable nanomicellar ophthalmic formulation of claim 30, wherein the phosphate buffer comprises sodium phosphate monobasic and/or sodium phosphate dibasic.

32. The stable nanomicellar ophthalmic formulation of claim 31, wherein the formulation comprises about 0.20 wt % to about 0.550 wt % sodium phosphate monobasic and about 0.23 wt % to about 0.465 wt % sodium phosphate dibasic.

33. The stable nanomicellar ophthalmic formulation of any one of claims 30 to 32, wherein osmolality of the formulation is about 100 to about 300 mOsmol/kg.

34. The stable nanomicellar ophthalmic formulation of any one of claims 30 to 33, wherein osmolality of the formulation is about 150 to about 200 mOsmol/kg.

35. The stable nanomicellar ophthalmic formulation of any one of claims 30 to 34, wherein pH of the formulation is about 6.5 to about 7.2.

36. The stable nanomicellar ophthalmic formulation of any one of claims 1 to 35, wherein density of the formulation is about 1.0 to about 1.5 g/ml.

37. The stable nanomicellar ophthalmic formulation of any one of claims 1 to 36, wherein surface tension of the formulation is about 30 mN/m to about 40 mN/m.

38. The stable nanomicellar ophthalmic formulation of any one of claims 1 to 37, wherein drop size of the formulation is about 20 μL to about 40 μL.

39. The stable nanomicellar ophthalmic formulation of any one of claims 1 to 38, wherein particle size Z_{avg} of the formulation is about 10 nm to about 20 nm.

40. The stable nanomicellar ophthalmic formulation of any one of claims 1 to 39, wherein zeta potential of the formulation is about 0 mV.

41. The stable nanomicellar ophthalmic formulation of any one of claims 1 to 40, wherein the formulation has about 100% cyclosporine encapsulation efficiency.

42. The stable nanomicellar ophthalmic formulation of any one of claims 1 to 41, wherein cloud point of the formulation is in about 40° C. to about 45° C.

43. The stable nanomicellar ophthalmic formulation of any one of claims 1 to 42, wherein the cyclosporine comprises any one of forms (i)-(iv):
(i) an amorphous form;
(ii) a form with characteristic XRD peaks at 2-theta (deg.) 6.9, 7.8, 9.4 and 15.9
(iii) a form with characteristic; XRD peaks at 2-theta (deg.) 7.4, 8.7, 14.4 and 17.5; and
(iv) a form with characteristic XRD peaks at 2-theta (deg.) 8.5, 9.3, 11.6 and 20.3.

44. The stable nanomicellar ophthalmic formulation of any one of claims 1 to 43, comprising less than about 2 wt % of an impurity characterized by relative retention time (RRT) of about 2.0 to about 2.7, wherein the RRT is determined by a method as described in the United States Pharmacopeia (USP) for cyclosporine.

45. A method of making a stable nanomicellar ophthalmic formulation, comprising:
a) mixing hydrogenated 40 polyoxyl castor oil and octoxynol-40 at a temperature of 100° C. or above, wherein the mixing produces a Mixture A comprising a water content of less than 3%;
b) adding cyclosporine to the Mixture A to form a Mixture B; and
c) mixing the Mixture B with the aqueous vehicle, thereby forming the stable nanomicellar ophthalmic formulation.

46. The method of claim 45, wherein the octoxynol-40 comprises less than 20 ppm total impurities.

47. A method of making a stable nanomicellar ophthalmic formulation, comprising:
a) mixing hydrogenated 40 polyoxyl castor oil and octoxynol-40, wherein the octoxynol-40 comprises less than 20 ppm total impurities and a water content of less than 10%, wherein the mixing produces a Mixture A comprising a water content of less than 3%;
b) adding cyclosporine to the Mixture A to form a Mixture B; and
c) mixing the Mixture B with the aqueous vehicle, thereby forming the stable nanomicellar ophthalmic formulation.

48. The method of claim 47, wherein the mixing of a) is at a temperature above 55° C.

49. The method of claim 48, wherein the mixing of a) is at a temperature above 100° C.

50. The method of any one of claims 45 to 49, wherein the octoxynol-40 comprises less than 10 ppm total impurities.

51. The method of any one of claims 45 to 50, wherein the octoxynol-40 comprises less than 5 ppm total impurities.

52. The method of any one of claims 45 to 51, wherein the method makes at least 20 L of the formulation.

53. The method of any one of claims 45 to 52, wherein the method makes at least 100 L of the formulation.

54. The method of any one of claims 45 to 53, wherein the method makes at least 1000 L of the formulation.

55. The method of any one of claims 45 to 54, wherein the Mixture B comprises a water content below about 3%.

56. The method of any one of claims 45 to 55, wherein the water content of Mixture A and/or the water content of Mixture B is below about 2%.

57. The method of any one of claims 45 to 56, wherein the water content of Mixture A and/or the water content of Mixture B is below about 1%.

58. The method of any one of claims 45 to 57, wherein the water content of Mixture A and/or the water content of Mixture B is below about 0.5%.

59. The method of any one of claims 45 to 58, wherein the mixing of a) is at a temperature of about 100° C. to about 200° C.

60. The method of any one of claims 45 to 59, wherein the mixing of a) is at a temperature of about 110° C. to about 160° C.

61. The method of any one of claims 45 to 60, wherein the mixing of a) is at temperature of about 120° C. to about 140° C.

62. The method of any one of claims 45 to 61, wherein the mixing of a) is at a temperature of about 127° C. to about 130° C.

63. The method of any one of claims 45 to 62, wherein the mixing of a) is at a temperature of about 130° C. or above.

64. The method of any one of claims 45 to 63, wherein the mixing of a) is performed for about 10 minutes to about 90 minutes.

65. The method of any one of claims 45 to 64, wherein the mixing of a) is performed for about 20 minutes to about 60 minutes.

66. The method of any one of claims 45 to 65, wherein the mixing of a) is performed for about 30 minutes to about 40 minutes.

67. The method of any one of claims 45 to 66, wherein the mixing of a) is performed at about 100 to about 800 rpm.

68. The method of any one of claims 45 to 67, wherein the mixing of a) is performed at about 130 to about 180 rpm.

69. The method of any one of claims 45 to 68, wherein step b) is performed at a temperature of 55° C. or above.

70. The method of any one of claims 45 to 69, wherein step b) is performed at a temperature of 100° C. or above.

71. The method of any one of claims 45 to 70, wherein step b) is performed at a temperature of 130° C. or above.

72. The method of any one of claims 45 to 71, wherein step b) is performed at the same temperature as the mixing of a).

73. The method of any one of claims 45 to 72, wherein step b) further comprises mixing until the cyclosporine is completely dissolved.

74. The method of any one of claims 45 to 73, wherein the aqueous vehicle comprises Water for Injection (WFI).

75. The method of any one of claims 45 to 74, further comprising, following step c), adding a buffer, a tonicity agent, a bioadhesive agent, a pH adjusting agent, or combination thereof.

76. The method of claim 75, wherein the buffer comprises phosphate buffer; the tonicity agent comprises sodium chloride, the bioadhesive agent comprises povidone; and the pH adjusting agent comprises sodium hydroxide and/or hydrochloric acid.

77. The method of any one of claims 45 to 76, further comprising, following step c):
adding in the following order: i) sodium phosphate monobasic; ii) sodium phosphate dibasic; iii) sodium chloride; and iv) povidone.

78. The method of claim 77, further comprising, following iv):
v) adjusting pH to about 6.5 to about 7.2 with sodium hydroxide and/or hydrochloric acid.

79. The method of any one of claims 45 to 78, wherein the stable nanomicellar ophthalmic formulation comprises:
0.09 wt % cyclosporine;
about 1.0 wt % hydrogenated 40 polyoxyl castor oil; and
about 0.05 wt % octoxynol-40.

80. The method of any one of claims 45 to 79, wherein the stable nanomicellar ophthalmic formulation comprises:
0.09 wt % cyclosporine;
about 1.0 wt % hydrogenated 40 polyoxyl castor oil;
about 0.05 wt % octoxynol-40;
about 0.05 wt % sodium chloride;
about 0.3 wt % povidone;
phosphate buffer; and
sodium hydroxide and/or hydrochloric acid.

81. The method of claim 80, wherein the phosphate buffer comprises sodium phosphate monobasic and/or sodium phosphate dibasic.

82. The method of claim 81, wherein the formulation comprises about 0.20 wt % to about 0.550 wt % sodium phosphate monobasic and about 0.23 wt % to about 0.465 wt % sodium phosphate dibasic.

83. The method of any one of claims 79 to 82, wherein osmolality of the formulation is about 100 to about 300 mOsmol/kg.

84. The method of any one of claims 79 to 83, wherein osmolality of the formulation is about 150 to about 200 mOsmol/kg.

85. The method of any one of claims 79 to 84, wherein pH of the formulation is about 6.5 to about 7.2.

86. The method of any one of claims 45 to 85, wherein density of the formulation is about 1.0 to about 1.5 g/ml.

87. The method of any one of claims 45 to 86, wherein surface tension of the formulation is about 30 mN/m to about 40 mN/m.

88. The method of any one of claims 45 to 87, wherein drop size of the formulation is about 20 μL to about 40 μL.

89. The method of any one of claims 45 to 88, wherein particle size Z_{avg} of the formulation is in the range of about 10 nm to about 20 nm.

90. The method of any one of claims 45 to 89, wherein zeta potential of the formulation is about 0 mV.

91. The method of any one of claims 45 to 90, wherein the formulation has about 100% cyclosporine encapsulation efficiency.

92. The method of any one of claims 45 to 91, wherein cloud point of the formulation is about 40° C. to about 45° C.

93. The method of any one of claims 45 to 92, wherein the cyclosporine comprises any one of forms (i)-(iv):
(i) an amorphous form;
(ii) a form with characteristic XRD peaks at 2-theta (deg.) 6.9, 7.8, 9.4 and 15.9
(iii) a form with characteristic; XRD peaks at 2-theta (deg.) 7.4, 8.7, 14.4 and 17.5; and
(iv) a form with characteristic XRD peaks at 2-theta (deg.) 8.5, 9.3, 11.6 and 20.3.

94. The method of any one of claims 45 to 93, wherein the formulation comprises less than about 2 wt % of an impurity characterized by RRT of about 2.1 to about 2.2, wherein the RRT is determined by a method as described in the United States Pharmacopeia (USP) for cyclosporine.
